(19) United States
(12) Patent Application Publication
(10) Pub. No.: US 2023/0149709 A1
(43) Pub. Date: May 18, 2023

(54) SYSTEM AND METHOD OF CONTROLLING BLADDER AND RECTAL FUNCTION

(71) Applicant: Regenerative Bioelectronics Inc., Worcester, MA (US)

(72) Inventors: Mario I. Romero-Ortega, Coppell, TX (US); David Constantine, Sudbury, MA (US)

(21) Appl. No.: 17/985,843

(22) Filed: Nov. 12, 2022

Related U.S. Application Data
(60) Provisional application No. 63/278,801, filed on Nov. 12, 2021.

(51) Int. Cl. A61N 1/36; A61N 1/05

(52) U.S. Cl. CPC A61N 1/36007; A61N 1/0558; A61N 1/36171; A61N 1/36175

(57) ABSTRACT

A method and/or system controls bladder function of a patient having a symptom related to overactive bladder (OAB) or stress unitary incontinence (SUI). The symptom related to OAB is produced by a natural OAB signal generated by the patient's body. To that end, the method couples an electrode to a prescribed somatic motor nerve (e.g., the perineal nerve) associated with the pelvic floor, and then transmits, via the electrode, an OAB control signal to the prescribed somatic motor nerve. The OAB control signal is configured to activate the pelvic floor in a prescribed manner to mitigate the effect of the natural OAB signal on the pelvic floor.

### PRIORITY

This patent application claims priority from provisional U.S. patent application No. 63/278,801, filed Nov. 12, 2021, entitled, “SYSTEM AND METHOD OF CONTROLLING BLADDER FUNCTION,” and naming Mario I. Romero-Ortega and David Constantine as inventors, the disclosure of which is incorporated herein, in its entirety, by reference.

### FIELD

Illustrative embodiments of the invention generally relate to bladder function and, more particularly, various embodiments of the invention relate to selective neuromodulation for controlling bladder function.

### BACKGROUND

Suffered by millions of people in the US and worldwide, overactive bladder (“OAB”) can cause debilitating urgent and frequent urination with or without urinary incontinence. Puzzling to many in the art, OAB is associated with nocturia, but in the absence of urinary tract infection or other obvious pathological conditions. The pathophysiology and etiology of idiopathic OAB remains unknown, but it seems to be related to bladder detrussor muscle dysfunction, or detrusor overactivity. The current pharmacological treatment includes oral anti-muscarinics or oral β3-adrenoceptor agonists. Such treatments have significant drawbacks as they have limited efficacy and tolerability, and cause significant side effects including dry mouth, dry eyes, constipation, tachycardia, and potential long-term cognitive effects. Consequently, studies have shown that the majority of OAB patients stop taking these drugs within 6-12 months of treatment.

Another urinary problem, known as “stress urinary incontinence” (“SUI”), occurs when urine leaks out with sudden pressure on the bladder and urethra, causing the sphincter muscles to open briefly. With mild SUI, pressure may be from sudden forceful activities, such as moderate exercise, sneezing, laughing or coughing. If SUI is more severe, however, urine may leak with less forceful activities, such as standing up, walking or bending over. Urinary “accidents” like this can range from a few drops of urine to enough to soak through your clothes.

Those in the art typically treat both OAB, SUI, and FI with different treatment regimens.

### SUMMARY OF VARIOUS EMBODIMENTS

In accordance with one embodiment of the invention, a method stimulates a nerve. The method provides a neuromodulation device having a channel that leads to a chamber having an electrode therein. A nerve is positioned at least partially inside the chamber. The nerve is stimulated by the electrode using a first set of parameters configured to provide an afferent control signal. The nerve is then stimulated by the electrode using a second set of parameters configured to provide an efferent control signal.

In various embodiments, stimulating the nerve using the first set of parameters produced an afferent sensory control signal that is configured to treat overactive bladder and/or fecal incontinence. The afferent sensory control signal has a frequency of between about 2 Hz and about 20 Hz. In various embodiments, the afferent sensory control signal has an amplitude of between about 0.4 mA and about 1.0 mA. The afferent sensory control signal may have a stimulation pattern with a pulse duration of between about 200 microseconds and about 300 microseconds. The pulse duration and frequency may define a stimulation pattern.

In various embodiments, a recurring stimulation pattern defines a treatment session. The afferent sensory control signal may be used for a treatment session time of between about 10 minutes and about 30 minutes, and a treatment session duty cycle of between about 10% and about 100%. In various embodiments, the treatment session duty cycle comprises delivering pulses for about 15 seconds, stopping delivery of pulses for about 2.5 minutes, and repeating the treatment session duty cycle for between the about 10 minutes and about 30 minutes. Furthermore, various embodiments may provide 3 to 5 stimulation treatments per day.

Among other things, the intensity of the stimulation may be sub-threshold. Stimulating the nerve using the efferent motor control signal may be configured to treat stress urinary incontinence or fecal incontinence. In various embodiments, second set of parameters produce an efferent motor control signal configured to treat SUI or FI. The efferent motor control signal may have a frequency of between about 40 Hz and about 80 Hz.

Among other things, the efferent motor control signal may have a sub-threshold amplitude of between about 0.5 mA and about 2 mA. The efferent motor control signal may also have a pulse duration of between about 250 microseconds and about 400 microseconds. In some embodiments, the efferent sensory control signal has a treatment session time of between about 10 minutes and about 13 minutes, and a treatment session duty cycle of between about 10% and about 100%. The stimulation treatment session may be provided 3 to 5 times per day.

In some embodiments, the nerve may be the perineal nerve. In other embodiments, the nerve may be the rectal nerve.

In accordance with another embodiment, a method stimulates a nerve to treat stress urinary incontinence. The method provides a neuromodulation device having a channel that leads to a chamber having an electrode therein. The method stimulates a perineal nerve positioned at least partially inside the chamber with the electrode using an SUI control signal. The SUI control signal has a frequency of between about 50 Hz and about 80 Hz, a threshold stimulation pulse of between about 0.5 mA and 2.0 mA, a pulse duration of between about 250 microseconds and about 400 microseconds, a total SUI session time of between about 10 minutes and about 13 minutes, and the total SUI session has a duty cycle of between about 5% and about 15%.

Stimulating the perineal nerve treats SUI. Additionally, the method may stimulate the perineal nerve positioned at least partially inside the chamber with the electrode OAB control signal after stimulating the perineal nerve using the SUI control signal. The OAB control signal having a frequency of between about 2 Hz and about 20 Hz, a sub-threshold stimulation pulse of between about 0.4 mA and 1.0 mA, a pulse duration of between about 200 microseconds and about 300 microseconds, a total OAB session time of between about 10 minutes and about 30 minutes, and the total OAB session may have a duty cycle of between about 100% and about 10%.

In some embodiments, the stimulation pulse may be bi-phasic.

In accordance with yet another embodiment, a method stimulates a nerve to treat overactive bladder. The method provides a neuromodulation device having a channel that leads to a chamber having an electrode therein. The method stimulates a perineal nerve positioned at least partially inside the chamber with the electrode using an OAB control signal, the OAB control signal having a frequency of between about 2 Hz and about 20 Hz, a sub-threshold stimulation pulse of between about 0.4 mA and 1.0 mA, a pulse duration of between about 200 microseconds and about 300 microseconds.

In various embodiments, the total OAB session time may be between about 10 minutes and about 30 minutes. The total OAB session may have a duty cycle of between about 100% and about 10%. In various embodiments, stimulating the perineal nerve treats OAB.

In accordance with yet another embodiment, a method stimulates a nerve to treat fecal incontinence. The method provides a neuromodulation device having a channel that leads to a chamber having an electrode therein. The method stimulates the inferior rectal nerve positioned at least partially inside the chamber with the electrode using an efferent FI control signal, the efferent FI control signal having a frequency of about 50 Hz and about 80 Hz, a stimulation pulse of between about 0.5 mA and about 2.0 mA, a pulse duration of between about 200 microseconds and about 300 microseconds, a total efferent FI session time of between about 10 minutes and about 30 minutes, and the total efferent FI session has a duty cycle of between about 100% and about 10%.

In various embodiments, the stimulation is a threshold stimulation and may be bi-phasic. The method may also stimulate the inferior rectal nerve positioned at least partially inside the chamber with the electrode using an afferent FI control signal having a frequency of between about 2 Hz and about 20 Hz, a sub-threshold stimulation pulse of between about 0.4 mA and 1.0 mA, and a pulse duration of between about 200 microseconds and about 300 microseconds. The afferent FI control signal may be after stimulating the inferior rectal nerve using the efferent FI control signal, or vice versa.

In various embodiments, the total afferent FI session time may be between about 10 minutes and about 30 minutes. The total afferent FI session may have a duty cycle of between about 100% and about 10%. Additionally, or alternatively, the method may also stimulate the inferior rectal nerve positioned at least partially inside the chamber with the electrode using an OAB control signal. The OAB control signal may have a frequency of between about 2 Hz and about 20 Hz, a sub-threshold stimulation pulse of between about 0.4 mA and 1.0 mA, and a pulse duration of between about 200 microseconds and about 300 microseconds.

In accordance with another embodiment of the invention, a method controls bladder function of a patient having a symptom related to overactive bladder (“OAB”) or stress unitary incontinence (“SUI”). The symptom related to OAB is produced by a natural OAB signal generated by the patient's body. To that end, the method couples an electrode to a prescribed somatic motor nerve (e.g., the perineal nerve) associated with the pelvic floor, and then transmits, via the electrode, an OAB control signal to the prescribed somatic motor nerve. The OAB control signal is configured to activate nerves and/or muscles in the pelvic floor in a prescribed manner to mitigate the effect of the natural OAB signal.

Among other ways, the electrode may be coupled with the nerve by directly connecting the electrode to the prescribed somatic motor nerve.

The OAB control signal may be transmitted by using a signal generator to produce and transmit the OAB control signal with the electrode. The OAB control signal may be transmitted using one or more of wired or wireless communication media.

The OAB control signal has a set of one or more prescribed specifications. For example, those prescribed specifications may include one or more of:
an amplitude of between about 0.4 milliamps and 1 milliamp,
each pulse having a pulse duration of between about 200 microseconds and 400 microseconds (when the OAB control signal is a periodic signal),
a frequency of between about 5 Hertz and 20 Hertz (when the OAB control signal is a periodic signal), and
a duration for transmitting the OAB control signal of no less than 10 minutes and for no longer than 30 minutes in a single session.

To manage SUI, the signal generator also may produce an SUI control signal to the prescribed somatic motor nerve via the electrode. The SUI control signal typically has a frequency that is different from the frequency of the OAB control signal. The SUI control signal preferably is configured to activate and, therefore, strengthen the pelvic floor when applied via the prescribed somatic motor nerve. To minimize and manage muscle fatigue during a single session, the SUI control signal may be transmitted after the OAB control signal.

In accordance with another embodiment, a system controls bladder function of a patient having a symptom related to overactive bladder (OAB) or stress unitary incontinence (SUI). As with the prior noted embodiment, the symptom related to OAB is produced by an OAB signal generated by the patient's body. To that end, the system has an electrode configured to couple with the perineal nerve, and a receive interface configured to receive an OAB control signal for stimulating sensory afferents in those nerves. In addition, the system also has

In various embodiments, a signal generator with a transmit interface configured to communicate with the receive interface of the electrode. The signal generator is configured to transmit a precise OAB control signal toward the electrode via the transmit interface and receive interface. The OAB control signal has a set of prescribed specifications to activate the pelvic floor in a prescribed manner to mitigate the effect of the natural OAB signal on the pelvic floor.

The system can be produced and distributed as a kit having the electrode and signal generator. Moreover, in some embodiments, the signal generator has memory for storing the set of prescribed specifications, which may include those discussed above.

Illustrative embodiments of the invention are implemented as a computer program product having a computer usable medium with computer readable program code thereon. The computer readable code may be read and utilized by a computer system in accordance with conventional processes.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIGS. 1A-8 schematically show a neuromodulation device configured in accordance with illustrative embodiments. It should be noted that the dimensions listed in these figures are illustrative and not intended to limit various embodiments.

FIGS. 9A-11C schematically show an alternative embodiment of neuromodulation device configured in accordance with illustrative embodiments. It should be noted that the dimensions listed in these figures are illustrative and not intended to limit various embodiments.

FIG. 12 is an anatomical drawing showing various nerves and anatomy relevant to illustrative embodiments.

FIG. 13 is a neuromodulation process in accordance with illustrative embodiments.

FIG. 14 is an anatomical drawing showing various nerves and anatomy relevant to illustrative embodiments.

FIG. 15 is a neuromodulation process in accordance with illustrative embodiments.

### DESCRIPTION OF ILLUSTRATIVE EMBODIMENTS

In illustrative embodiments, a control system activates a somatic motor nerve in a prescribed manner to treat one or both overactive bladder (“OAB”) and/or stress urinary incontinence (“SUI”). To those ends, using neuromodulation techniques in an unexpected manner, a signal generator is configured to coordinate with an electrode directly coupled with a somatic motor nerve to stimulate the nerve in a prescribed manner to treat or improve symptoms of one or both of OAB and SUI. Accordingly, the signal generator is configured to generate and transmit one or more signals with precise specifications to accomplish those goals. In various embodiments, the selected somatic motor nerve preferably is the perineal nerve. Details of illustrative embodiments are discussed below.

Furthermore, the control system activates a somatic motor nerve in a prescribed manner to treat one or both overactive bladder (“OAB”) and/or fecal incontinence (“FI”). To those ends, using neuromodulation techniques in an unexpected manner, a signal generator is configured to coordinate with an electrode directly coupled with a somatic motor nerve to stimulate the nerve in a prescribed manner to treat or improve symptoms of one or both of OAB and FI. Accordingly, the signal generator is configured to generate and transmit one or more signals with precise specifications to accomplish those goals. In various embodiments, the selected somatic motor nerve preferably is the inferior rectal nerve. Details of illustrative embodiments are discussed below. Details of illustrative embodiments are discussed below.

In accordance with various embodiments, improving symptoms for overactive bladder results in a reduction of 50% or more in the number of episodes of urinary urgency and/or mean number of urinary urgency incontinence over a time period. The time period may be, for example, 24-hours, 1-week, and/or a month.

In accordance with various embodiments, improving symptoms for stress urinary incontinence results in a reduction of 50% or more in the number of episodes of involuntary urine leakage with cough stress test, and an increase in micturition volume (e.g., of 40% or more) over a time period. The time period may be, for example, 24-hours, 1-week, and/or a month.

In accordance with various embodiments, improving symptoms for fecal incontinence results in a reduction of 50% or more episodes of uncontrolled staining, solid or liquid fecal incontinence over a time period. The time period may be, for example, 24-hours, 1-week, and/or a month.

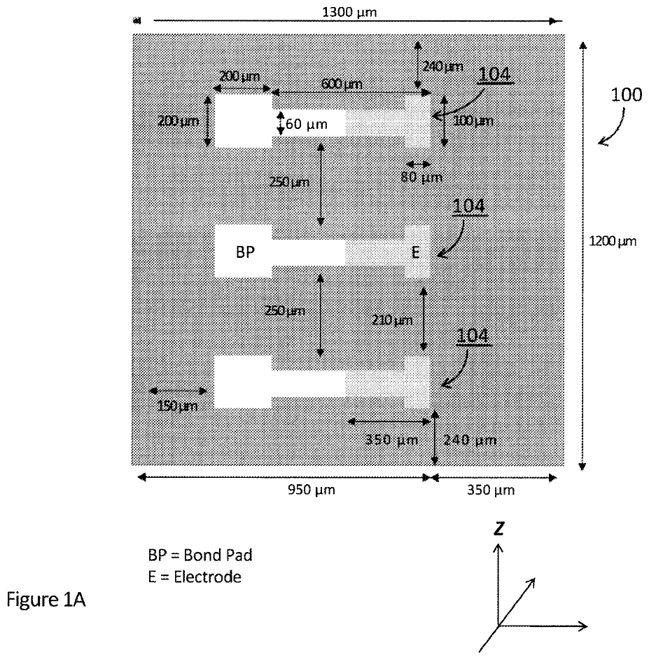

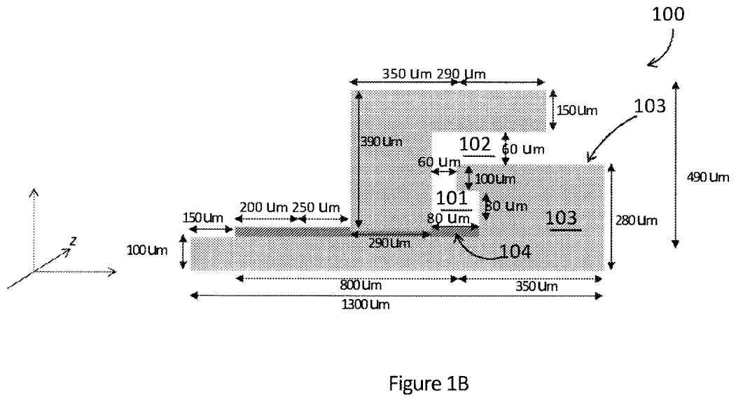

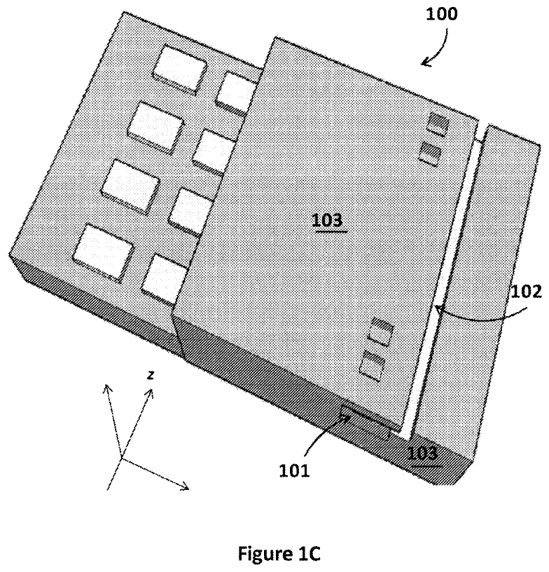

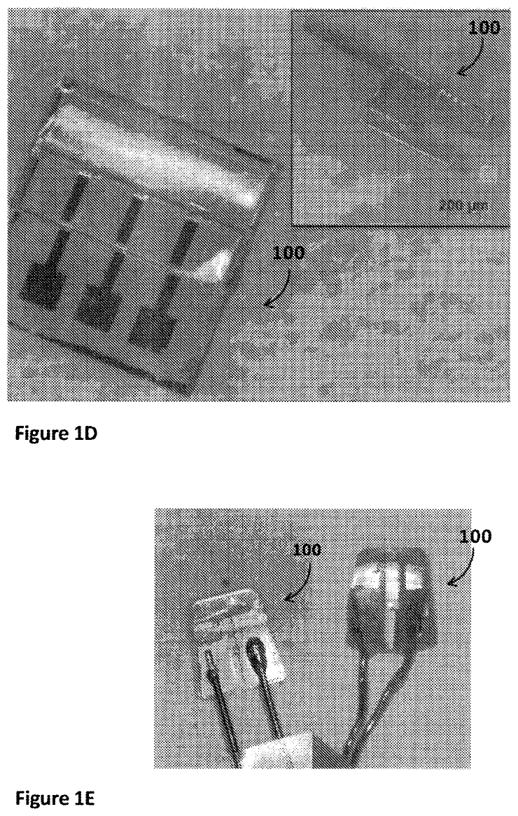

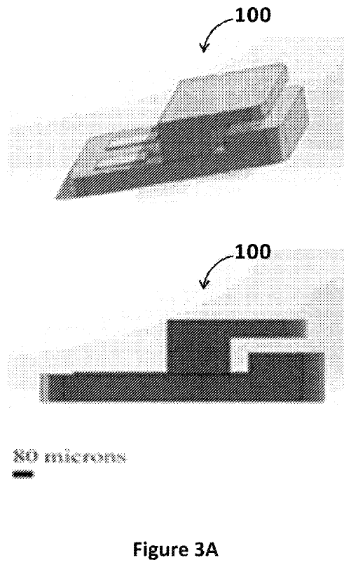

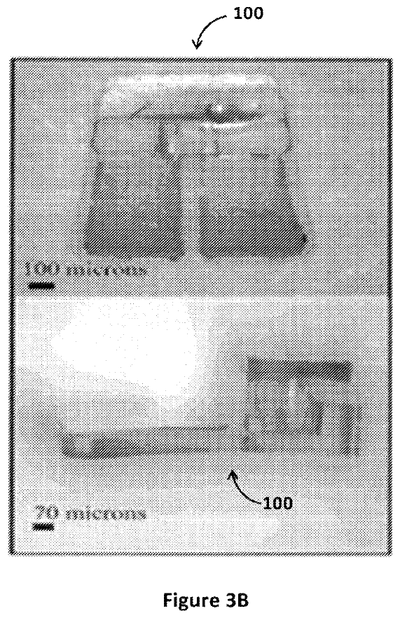

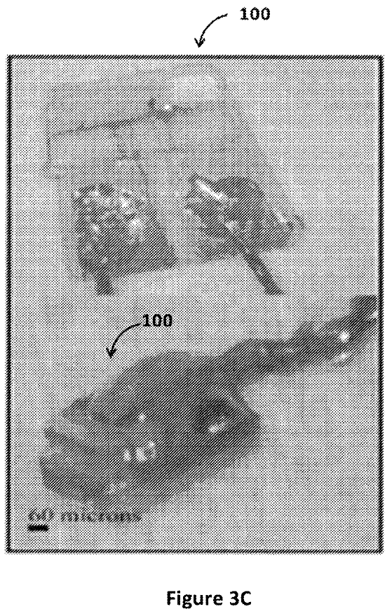

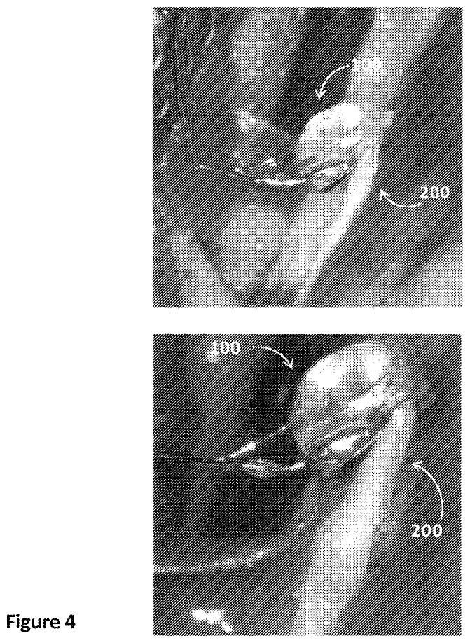

FIGS. 1A-4 schematically show a neuromodulation device 100 configured in accordance with illustrative embodiments. To that end, the neuromodulation device 100 has a housing forming an open chamber 101 configured to receive a nerve 200, at least one electrode 104 positioned in the chamber 101 to stimulate the nerve 200, and a channel 102 defined by two walls of the housing. In some embodiments, the housing forms the channel 102 to be in fluid communication with both the interior of the chamber 101 and the external surface 103 of the device 100. In some instances, the channel 102 can be in fluid communication with more than one external surface 103 of the device 100. In some embodiments, the channel 102 has an average width that is 5 percent to 50 percent smaller than the average width or other dimension of the chamber 101. The channel 102 can be linear, non-linear, curved, or irregularly shaped.

The channel 102 may have a central axis that extends from the proximal end to the distal end. The central axis may change direction from the proximal end to the distal end. In some embodiments, the channel cross-sectional inner dimension may be fixed (e.g., particular at the distal end that leads to the chamber 101). Additionally, the proximal end may be open to outside of the device 100, and the distal end may terminate at the chamber 101. In various embodiments, the channel 102 cross-sectional inner dimension at the distal end is less than a cross-sectional inner dimension of the chamber 101.

The device 100 may be formed at least in part from a polymer and selected metals. For example, in some embodiments, the neuromodulation device 100 is fabricated using flexible polyimide/Sic substrates with gold metallization in ultra-micro scale using established thin-film and photolithography methods. In another example, the device 100 can be formed from a glass substrate, silicone, insulated polyimide or other such polymer, using commonly employed microfabrication and photoresist techniques.

As noted above, the device 100 has conductive components. For example, the device 100 can be connected to an electrical pulse generator and/or an electrical stimulator (sometimes generically referred to as a “signal generator”). The device 100, in some cases, can comprise circuitry to communicate with the signal generator. For example, communication circuitry can facilitate magnetic inductive coupling or a direct conductive connection (e.g., via a wire or other hard-wired electrical interface).

In some embodiments, the chamber 101 can have the function of a recording chamber 101 and/or a stimulating chamber 101. For example, the recording chamber 101 can record electrical activity within the chamber 101 and the stimulating chamber 101 can elicit an electrical stimulus within the chamber 101.

The chamber 101 is configured to receive the nerve 200 (e.g., a human somatic motor nerve). The nerve 200 can include multiple nerve axons, a nerve fiber, a nerve bundle, a nerve fascicle, or other similar neuroanatomical structure. It should be understood, however, that the nerve 200 preferably is a functionally intact nerve or a partially-functional nerve. For example, a functionally intact nerve should have a functional pre- and post-synaptic terminal and should be functionally capable of propagating an action potential. For example, the nerve 200, in some embodiments, can have an average diameter of at least 50 μm (micrometers), at least 100 μm, at least 200 μm, at least 300 μm, at least 400 μm, at least 500 μm, at least 600 μm, at least 700 μm, at least 800 μm, or at least 900 μm. In some embodiments, the nerve 200 can have an average diameter between about 50 μm and 4 mm, between about 50 μm and 800 μm, or some other range between the noted exemplary sizes discussed above.

The chamber 101, in some embodiments, can be generally cylindrical in shape where the ends of a cylindrically shaped chamber 101 are open to permit longitudinal exit of the nerve 200 from the chamber 101 toward the pre- and post-synaptic terminals of the nerve 200. Whereas a cylinder comprises a circular cross-sectional shape, it should be understood that the chamber 101 can also comprise a triangular, square, pentagonal, hexagonal, or polygonal cross-sectional shape having n number of sides, while maintaining a general 3-dimensional structure resembling a cylinder, or a pipe, having open ends and operable to receive the nerve 200. Some embodiments of the chamber 101 have no specific cross-sectional shape and instead may be irregularly cross-sectionally shaped.

The chamber 101, in some embodiments, can be in fluid communication with an external surface 103 and a second (opposite) external surface 103 of the device 100. Specifically, the first and second external surfaces 103 in this example are on opposite sides of the device 100 and the chamber 101, positioned between the first and second external surfaces and in fluid communication with each opposing first and second external surfaces 103 of the device 100.

As a three-dimensional region, the chamber 101 has a length, width, and depth. The length of the chamber 101 corresponds to a z-axis that traverses longitudinally along the nerve 200 extending through the device 100, while either of the width and depth may correspond to the x-axis and y-axis. In some embodiments, the chamber 101 can have an average length of at least 10 μm. In some embodiments, the chamber 101 can have an average length of at least 50 μm, at least 100 μm, at least 500 μm, or at least 1000 μm. In some embodiments, the chamber 101 can have an average length of between about 10 μm and 11 mm. In some embodiments, the chamber 101 can have an average length of between about 10 μm and 5 mm, between about 10 μm and 3 mm, between about 10 μm and 1 mm, or between about 10 μm and 11 mm.

As noted, the width and depth of the chamber 101 can be considered to correspond to cross-sectional dimensions of an x-y-plane orthogonal to the z-axis of the chamber 101. For example, a cylindrically shaped chamber 101 can have a width and depth corresponding to a diameter of the chamber 101. The diameter of a non-cylindrically shaped chamber 101 can be measured by averaging the distance of measurements intersecting the center point of a cross-section of the chamber 101, wherein the center point is positioned on the z-axis extending through the middle of the chamber 101. In some embodiments, the chamber 101 can have an average diameter of about less than 10 mm. In some embodiments, the chamber 101 has an average diameter of about 10 μm (micrometers) to about 2000 μm, about 10 μm to about 4000 μm, 10 μm to about 3000 μm, about 1 μm to 2000 μm, about 10 μm to 1000 μm, about 10 μm to 900 μm, or about 10 to 800 μm, or about 10 μm to 500 μm.

In some embodiments, the chamber 101 has an average diameter that is substantially the same or ten percent smaller than the average diameter of a target nerve 200. In some embodiments, the average diameter of the chamber 101 is no more than 5 percent larger or no more than 5 percent smaller than the average diameter of the target nerve 200. In some embodiments, the average diameter of the chamber 101 is no more than 15 percent larger or no more than 15 percent smaller than the average diameter of the target nerve 200. For example, for a target nerve 200 having an average diameter of about 80 μm, the device 100 can have an average diameter of no less than about 56 μm, and no more than 104 μm. In some embodiments, the chamber 101 has an average diameter that is about 80-120 percent of a target nerve 200, about 85-115 percent of a target nerve 200, about 90-110 percent of a target nerve 200, about 95-105 percent of a target nerve 200, or about 100 percent or equal in size of a target nerve 200.

In some embodiments, the channel 102 is defined by two walls that can provide upper boundary and lower channel boundaries. In some instances, the distal end of the channel 102 can be in fluid communication with the interior of the chamber 101 and the proximal end of the channel 102 can be in fluid communication with the exterior surface 103 of the device 100. Thus, the distal end of the channel 102 is open to the chamber 101. In some embodiments, the chamber 101 is indefinitely or constantly open to the channel 102, such that the distal opening of the channel 102 into the chamber 101 does not close. Moreover, the channel 102 can connect the interior of the chamber to the external surface 103 of the device 100. Thus, in this embodiment, the chamber 101 is essentially in constant communication with an exterior surface 103 of the device via the channel 102. For example, the chamber 101 remains open to the channel at all times and the channel 102 remains open to an exterior surface at all times. As a three-dimensional region, the channel 102 has a length, a depth, and a diameter, which are not interchangeable. Similar to the length of the chamber 101 described above, a length of the channel 102 corresponds to a measurement along a z-axis, which traverses longitudinally along the nerve 200. The length can be measured at any point along the channel 102 between the distal end of channel opening into the chamber 101 and the proximal end of a channel opening to an exterior surface 103 of the device. In some embodiments, the distal end of the channel 102 can be in fluid communication with the chamber 101 for the entire length of the chamber 101. In some cases, the average length of the channel 102 is substantially the same as the average length of the chamber 101 of the device 100 described herein.

In some embodiments, the channel 102 can have an average length of at least 100 μm. In some embodiments, the channel 102 can have an average length of at least 1000 μm, at least 2000 μm, or at least 4000 μm, or at least 6000 μm. In some embodiments, the channel 102 can have an average length of between about 100 μm and 10 mm. In some embodiments, the channel 102 can have an average length of between about 100 μm and 6 mm, between about 100 μm and 3 mm, between about 100 μm and 8 mm, or between about 10 μm and 9 mm (e.g., an embodiment of the device with a battery).

The depth of the channel 102 corresponds to a distance measured between the distal opening and the proximal opening of the channel 102, wherein the distance is measured along an imaginary centerline positioned equidistant between each channel wall. In some cases, a depth can be a linear measurement. For example, in some cases, the channel 102 is a linear channel 102. In other cases, the channel 102 can be non-linear, wherein a non-linear channel comprises one or more turns, curves, or bends in the channel walls. Thus, in some instances, the depth of a non-linear channel 102 can be measured by measuring the distance along the imaginary centerline of the channel 102 between the distal opening and proximal opening of the channel 102, and along each bend in the non-linear channel 102. For example, in some embodiments, the channel 102 can comprise an “L” shape, such that the channel 102 depth measurement comprises a 90-degree turn and each end of the “L” corresponds to the distal and proximal openings of the channel. In an exemplary channel having a 90-degree turn, the depth can be measured by summing the distance of an imaginary centerline of the channel for each arm in the “L” of the channel 102 extending between the proximal opening and the distal opening of the channel 102 to where the imaginary lines of each arm meet. The shape of the channels can also include other configurations such as T, Z and S, and others.

In some embodiments, the channel 102 can have an average depth of between about 100 μm and 5 mm (e.g., 4.5 mm). In some embodiments the channel 102 can have an average depth of between about 100 μm and 5 mm, between about 100 μm and 1 mm, between about 100 μm and 2 mm, between about 100 μm and 3 mm, between about 100 μm and 4 mm, or between about 100 μm and 5 mm.

The diameter of the channel 102 corresponds to a measurement of the channel 102 positioned in an x-y plane that is orthogonal to the z-axis, as described above. The diameter of the channel 102 can be constant, such that the diameter of the channel 102 does not change between the proximal opening and distal opening of the channel 102. That is, in some embodiments, the diameter of the channel 102 comprises less than 10 percent variability of an average diameter across an entire depth of the channel 102. In some cases, the channel 102 comprises less than 5 percent variability, less than 3 percent variability, or less than 2 percent variability of an average diameter along an entire depth measurement of the channel 102. In some cases, a diameter can be determined by measuring the shortest distance between the two walls of the channel 102.

The diameter of the channel 102, in some embodiments, is less than the diameter of a target nerve 200. For example, in some embodiments, the channel 102 diameter can be at least 5 percent smaller than a diameter of a target nerve 200. In some embodiments, the channel 102 diameter can be at least 5 percent, at least 10 percent, at least 15 percent, at least 20 percent, at least 25 percent, at least 30 percent, at least 35 percent, at least 40 percent, at least 45 percent, or at least 50 percent smaller than a diameter of the nerve 200. In some embodiments, the channel 102 diameter can be no more than 60 percent smaller than a target nerve 200 diameter. In other embodiments, the channel 102 diameter can be no more than 50 percent smaller than a target nerve 200 diameter. In some embodiments, the channel 102 diameter can be between about 5 percent and 60 percent smaller than a diameter of a target nerve 200. In some cases, the channel 102 diameter can be between about 10 percent and 50 percent, between about 10 percent and 40 percent, between about 15 percent and 40 percent, between about 20 percent and 35 percent, or between about 20 percent and 40 percent smaller than a diameter of a target nerve 200.

Furthermore, similar to the chamber 101 described above, the channel 102 can have open ends in fluid communication with the open ends of the chamber 101 such that the nerve 200 can be inserted into the chamber 101 by sliding, moving, or inserting a longitudinal section of the nerve 200 into the chamber 101 via the channel 102. Thus, the channel 102 is configured to receive the target nerve 200. Moreover, in some embodiments, the channel 102 can be in fluid communication with one, two, or all three external surfaces 103 of the device 100. For example, the channel 102 can be open to an interior of the chamber 101 at the distal end of the channel and the channel can extend along the depth of the channel to the third external surface 103 at the proximal end of the channel, while maintaining fluid communication with the first external surface 103 and the second external surface 103 on opposing sides of the device 100 corresponding to opposing ends of the z-axis.

As noted above, the device 100 has at least one electrode 104 within the chamber 101. The electrode 104 can include various types of electrodes, including, among other types, fiber or flat electrodes, thin film electrodes, or needle electrodes. As an example, the electrode 104 can be implemented as a thin film electrode with a recording or stimulating surface within 100-2000 μm2 or within 50 μm of an outer surface of the nerve 200 when coupled within the chamber 101. A needle electrode in the chamber can have a needle shaped recording and/or stimulating surface that can penetrate the surface of the nerve 200 when in the chamber 101. The electrode that penetrates a nerve in the chamber 101 can stimulate and/or record intraneurally, which can provide greater selectivity and/or resolution when recording and/or stimulating. Additionally, the electrode 104 can be positioned on any of a number of other surfaces within the chamber 101, including the top, a bottom, and/or side chamber surfaces.

A combination of electrode types can be used when the chamber 101 has more than one electrode 104. For example, both flat electrodes and/or needle electrodes can be used in a recording and/or stimulating chamber 101. Among others, the electrode(s) 104 can be mono-polar, bi-polar, tri-polar, or a multi-electrode array electrodes. In some cases, a plurality of electrodes 104 can be configured in a tripolar configuration, which should provide improved nerve specificity and/or selectivity while simultaneously reducing extraneous biological noise.

As noted above, the electrode 104 can be formed at least in part from one or more conductive metals. For example, the electrode can be formed at least in part from gold, titanium nitride (TiN), iridium oxide (IrO), iridium, carbon nanotubes, graphene, graphene oxide, and/or platinum (Pt). The electrode 104, in some instances, can have a charge injection capacity of about 0.1 mC/cm2 or greater. Further, in some embodiments, the electrode 104 can be a wired or a wireless electrode. A wireless electrode 104 can have a wireless integrated circuit that enables communication with external devices.

In some embodiments, the electrode 104 can have a stimulating and/or recording surface area of between about 25 μm2 and 25 mm2. In some instances, the electrode comprises a stimulating and/or recording surface area of between about 100 μm2 and 1 mm2 or between about 100 μm2 and 0.5 mm2.

Illustrative embodiments use prescribed neuromodulating techniques to selective activate one or more pelvic floor muscles. Among others, those pelvic floor muscles may include the cremaster muscle, bulboglandularis muscle (Bgm), ischiocavernosus muscle (Ism), bulbospongiosus muscle (Bsm), pubococcygeus muscle (Pcm), iliococcygeus muscle (Icm), coccygeus muscle (Cgm), or puborectalis muscle (Prm). Preferably, however, the pubococcygeus muscle is actuated using the perineal nerve. In some embodiments, the pelvic floor muscle can be neuromodulated or stimulated simultaneously or independently of one or more other pelvic floor muscles. In some instances, a pelvic nerve can include any nerve, nerve bundle, nerve fascicle, or nerve tract that innervates a pelvic floor muscle or pelvic organ, including the pudendal nerve, the clitoralis nerve or the dorsal nerve of the penis, or any branch of these or other nerves in the pelvis.

It should be understood than the device 100 discussed above is but one example of a neuromodulation device that can be used to stimulate the appropriate somatic nerve. For example, two or more devices 100 can be used to stimulate two or more pelvic floor muscles or organs. Furthermore, since the device 100 can record and/or stimulate, two devices can be used on the same pelvic floor muscle to independently record and stimulate, or one device can be used to record and stimulate the pelvic floor muscle or organ.

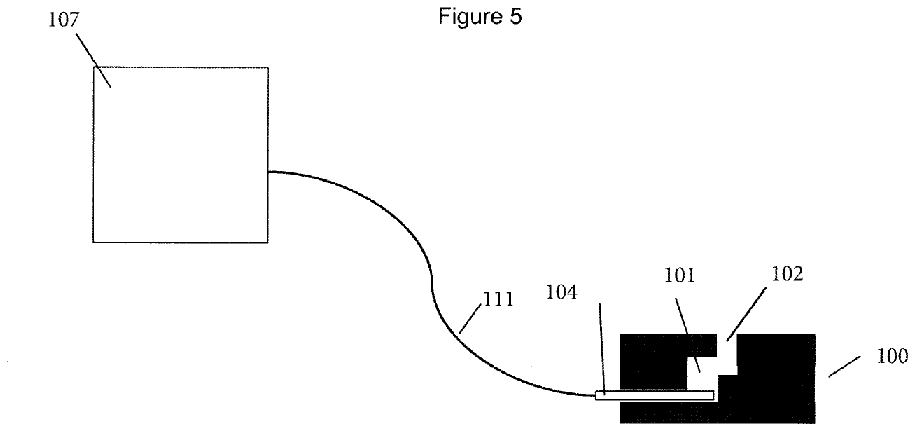

FIG. 5 schematically shows another embodiment of the device 100 in accordance with illustrative embodiments. Note that this figure has different reference numbers for the same components from those above, but similar components have the same or similar functionality. As shown, the device includes a battery or otherwise powered pulse generator and electronic controller 107 connected to an electrode 104 by way of a conductive wire 111. As illustrated, the electrode is a part of the chamber 101 of the neuromodulation device 100. As illustrated, the neuromodulation device 101 may include a “L” shaped channel 102 configured to receive a nerve. In some embodiments, at least a portion of the diameter of the channel 102 may be smaller than the diameter of the target nerve. Accordingly, the target nerve may be temporarily reversibly compressed or stretched, and slid along the channel until the target nerve is held within the chamber 101. In some embodiments, the chamber 101 may have a diameter greater than the nerve, such that the nerve is not compressed or stretched within the chamber 101. Alternatively, the chamber 101 may have a diameter smaller than the nerve. Accordingly, in such an embodiment, at least a portion of the nerve may extend into the channel 102 while a substantial portion of the nerve is contained within the chamber 101. Further, the chamber 101 may provide an isolated fluidic environment that allows for the targeted and specific stimulation of the portion of the nerve held within the chamber 101.

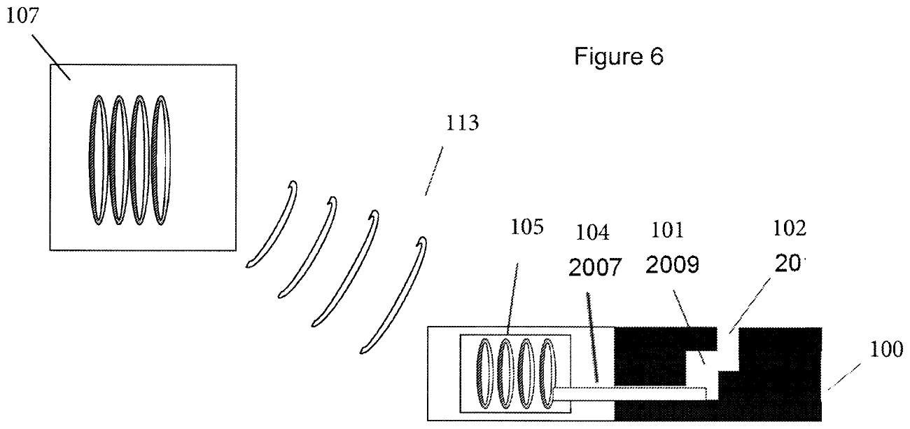

FIG. 6 schematically shows a wireless neuromodulation device and external system in accordance with illustrative embodiments. As with FIG. 5, this figure has different reference numbers for the same components from those above, but similar components have the same or similar functionality. The device has an external battery or otherwise powered pulse generator and electronic controller 107 that includes a coil to transmit power, data and/or control signals to a wireless neuromodulation device 100. In particular, an electromagnetic field 113 may couple the pulse generator and electronic controller 107 to the wireless neuromodulation device 100. Corresponding electronics and a magnetic induction coil 105 in the neuromodulation device 100 may be connected to the conductive material used as electrode(s) 104. The electrodes 104 may form part of the chamber 101. As discussed with other embodiments, the neuromodulation device of this figure may include a chamber 101 configured to receive the nerve, and a channel 102 (e.g., an “L-shaped slit”) through which the nerve may pass as it is placed in the chamber 101.

FIG. 7 schematically shows another wireless embodiment of the system. An external battery powered pulse generator and electronic controller 107 with a coil is configured to transmit power, data and/or control signals to a neuromodulation device 100. An electromagnetic field 2103 couples with the coil 105 of the neuromodulation device 100. In some embodiments, the electronics and coil 105 may be spaced apart and implanted separately from the stimulating elements of the neuromodulation device 100. The location of the electronics and coil 105 may be chosen to optimize the signal strength and quality of transmissions between the external pulse generator and electronic controller 107 and the neuromodulation device 100.

Further, the separate electronics and coil 105 configuration may lead to reduced battery requirements and applied voltage and/or amplitude. A conductive wire 111 may couple the electronics and coil 105 to the neuromodulation device 100, and more particularly to the electrodes 104 which may be located within a chamber 101. As with some other embodiments, the neuromodulation device 100 may include a chamber 101 configured to receive the nerve, and a channel 102 (or “L-shaped slit”) through which the nerve may pass as it is placed in the chamber 101.

Among other benefits, connecting implanted electronics with a receiver coil to the neuromodulation device effectively separates the receiver coil. This enables a medical practitioner to implant the receiver coil in the same location and orientation relative to the hard tissue structures of the body regardless of where the electrode and neuromodulation device is positioned. This consistency in the location and orientation of the receiver coil is expected to provide a more consistent, efficient and reliable coupling and induction in the implanted coil and electronics. This may help avoid variability in stimulation or variability in external coil position or orientation requirements.

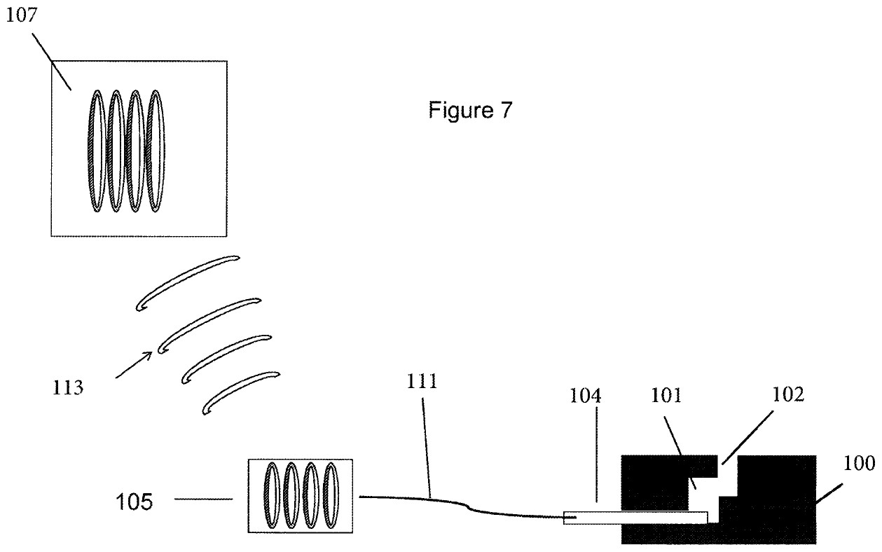

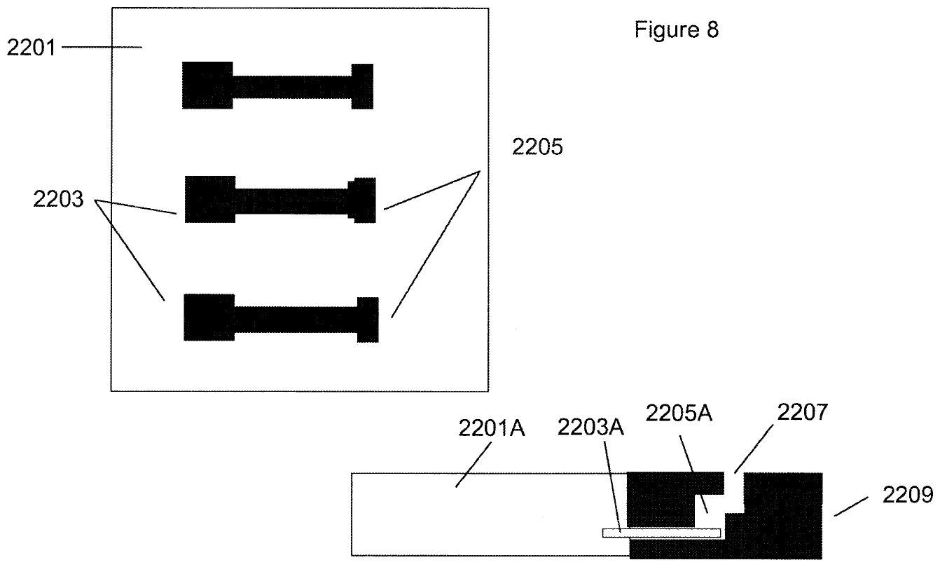

FIG. 8 schematically shows another embodiment of a neuromodulation system. FIG. 8 has different reference numbers for the same components from those above, but similar components have the same or similar functionality. This figure shows a bottom view 2201 of the neuromodulation device and a side view of the overall device. As shown, the neuromodulation device includes insulated material that forms the body of the device, and conductive material 2203 forming one or more electrode(s) with contact pads to connect to wires of wireless electronics, and a contact pad for nerve stimulation/recording 2205. The side view 2209 of the neuromodulation device illustrates the insulated material that forms the body of the device 2201A, as well as the conductive material used as electrode(s) 2203A. This view 2209 also shows a chamber 2205A where the nerve is placed after implantation, and the channel 2207 through which the nerve is inserted or engaged with the neuromodulation device 2209.

Alternative embodiments, may include a neuromodulation device shaped in a different manner (e.g., as a clothespin, dovetail, and/or vase). Each of the disclosed designs may be configured to include a channel having a diameter smaller than that of the target nerve such that the nerve is stretched prior to engaging with the chamber of the neuromodulation device.

Those skilled in the art should understand that a variety of neuromodulation devices 100 are suitable for use with the methods described herein. For example, devices 100 and materials described in U.S. provisional patent applications 63/391,584, and 63/391,574, which are both incorporated herein by reference in their entireties, are suitable for use with the methods described herein. The devices described in the incorporated applications describe suitable alternative neuromodulation devices 100, an example of which is described below.

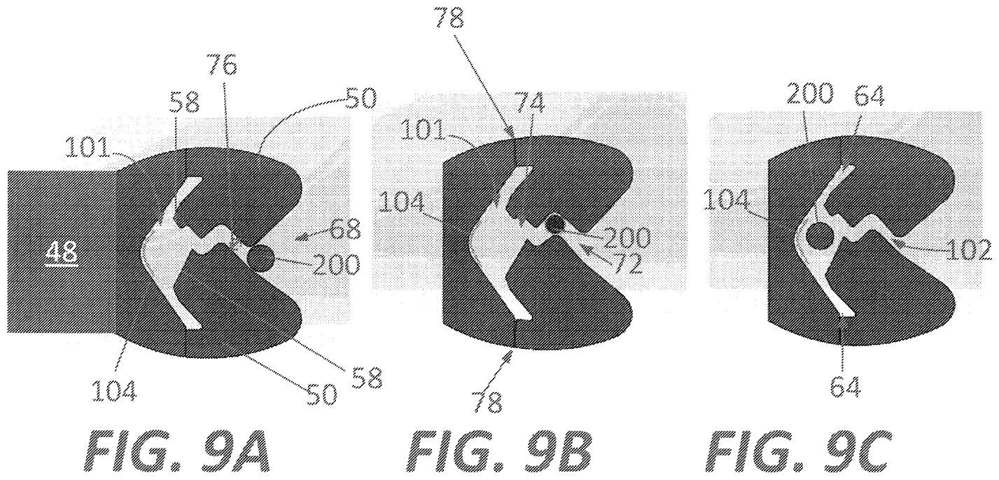

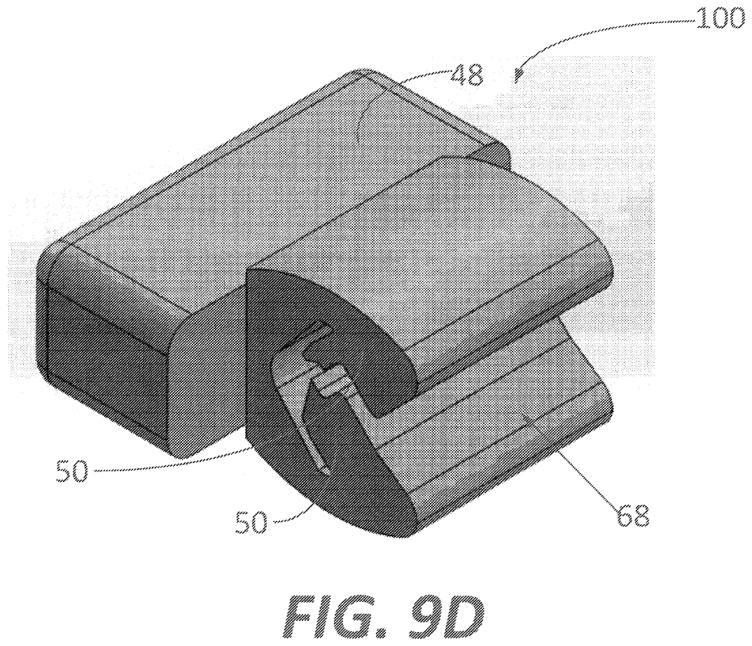

FIGS. 9A-11C schematically show an alternative embodiment of the neuromodulation device 100 in accordance with illustrative embodiments. Specifically, FIGS. 9A-11C show an embodiment having a plurality of jaws 50 (e.g., formed from silicone or including a silicone overmold). The main body 40 is omitted in some of these figures, but it should be understood that various embodiments may include the main body 40, the housing 48 (e.g., ceramic), and/or other components described previously. However, contrary to previously shown embodiments having the channel 102 oriented substantially perpendicular to the main body 40, FIGS. 9A-11C show the channel 102 oriented substantially horizontal to the main body 40. However, in other embodiments, the channel 102 may be oriented in any direction (e.g., diagonal to the main body 48).

Advantageously, a single device 100 is configured to couple with and stimulate nerves 200 of a variety of sizes (e.g., between 0.5 mm and about 4 mm).

Although the main body 48 is shown in FIG. 9A as being smaller than the jaws 50, it should be understood that the drawing is not necessarily to scale. In some embodiments, the main body 48 may be larger or smaller than the jaws 50. Additionally, the main body 48 may form part of the chamber 101 having the electrode 104. In some other embodiments, however, the main body 48 may not form part of the chamber 101. Instead, the jaws 50 and/or an intermediary between the jaws 50 may define the chamber 101.

FIGS. 9A-9C schematically show a process of positioning a small nerve 200 (e.g., about 0.5 mm to about 1.5 mm in diameter) within the chamber 101 for stimulation. The figures show a side view of the device 100.

For the sake of discussion, the nerve shown in FIGS. 9A-9C is a small nerve of about 1 mm in diameter. Together, the jaws 50 define a V-shape funnel 68 on the exterior of the device 100 that is configured to assist a surgeon with positioning the nerve 200 at the proximal end 72 of the channel 102. When the nerve 200 is positioned at the proximal end 72 of the channel 102, the nerve 200 preferably has a greater cross-sectional dimension than the channel 102. For example, the channel 102 may have a cross-sectional dimension of between about 0.45 mm and about 0.75 mm. However, in some embodiments, the channel 102 may be completely closed (e.g., a dimension of about 0 mm). Advantageously, the reduced channel dimension 76 prevents accidental dislodgement of the nerve 200. Additionally, to that end, the channel 102 may have non-linear or tortuous shape to assist with preventing accidental dislodgement of the nerve 200. However, some embodiments may include a linear channel 102.

In any event, as shown in FIG. 9B, the nerve 200 may be stretched (e.g., by the surgeon applying a force) and/or squeezed (e.g., by the inner surface of the channel 102) to reduce the cross-sectional dimension of the nerve 200. Preferably, the reduction in nerve 200 dimension is less than 50% to prevent or reduce damage to the nerve 200 that might otherwise occur from greater reductions in cross-sectional size. For example, the nerve 200 may be reduced to a cross-sectional dimension of about 0.75 mm. Assuming the channel dimension 76 is about 0.5 mm, the nerve 200 may still be too large to pass through the channel 102. To that end, the device 100 may include hinges 78 that allow the jaws 50 to open and thereby expand the channel dimension 76. In some embodiments, the hinges 78 are formed by a deformable material (e.g., silicone) that allow the jaws 50 to open outwardly. Additionally, or alternatively, the channel 102 may include a deformable and/or resilient wall or coating that provide for expanding the channel dimension 76.

As shown in FIG. 9C, when the nerve 200 passes the distal end 74 of the channel 102 and enters the chamber 101, the jaws 50 may again close. Because of the reduced channel dimension 76 and the non-linear channel 102 shape, the small nerve 200 is securely coupled within the chamber 101. Although not drawn to scale, the jaws 50 may include nerve contact surfaces 58 configured to press and/or hold the nerve 200 against the electrode 104. In various embodiments, the jaws 50 may be biased to return to a close position, and/or the deformable material may be resilient and return to its original dimensions.

FIG. 9D schematically shows a perspective view of the device 100 (with details of the chamber 101, such as the electrode 104 omitted).

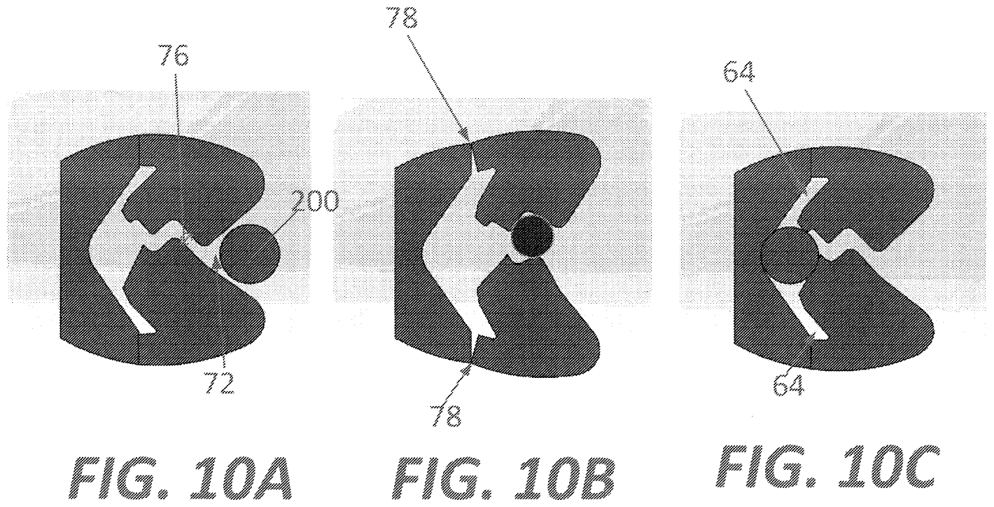

FIGS. 10A-10C schematically show a process of positioning a medium nerve 200 (e.g., about 1.5 mm to about 2.5 mm in diameter) within the chamber 101 for stimulation. The figures show a side view of the device 100.

For the sake of discussion, the nerve 200 shown in FIGS. 10A-10C is a medium nerve of about 2 mm in diameter. Similar to the process described previously, the V-shape funnel assists with positioning the nerve 200 relative to the proximal end 72 of the channel 102. For the sake of discussion, the nerve 200 may be stretched to reduce the nerve diameter to about 1.5 mm. As described previously, the channel dimension 72 may be about 0.5 mm. Accordingly, the nerve is considerably thicker than the channel 102 is wide when the jaws 50 are in the resting position. The jaws 50 may be expanded open to increase the channel dimension 76 and to allow the nerve 200 to pass through the channel. To that end, illustrative embodiments may include a delivery device with cam features that press the jaws 50 open via deformation and/or hinging. Although FIG. 9B schematically shows the hinge splitting open, various embodiments do not have a hinge opening (which might otherwise trap a portion of a larger deformed nerve).

As shown in FIG. 10C, the nerve 200 comes to rest in the chamber 101 and the jaws 50 may be reclosed. For example, the nerve 200 relaxes and returns to its 2 mm diameter. Additionally, the silicone of the jaws 50 may relax and the channel dimension 76 is restored to about 0.5 mm, preventing the nerve from backing out of the channel 102. In some embodiments, the jaws 50 are biased to a resting position (e.g., where the channel 102 has a dimension 76 of about 0.5 mm or to a fully closed position). Additionally, or alternatively, the delivery device may close the jaws 50. The silicone deformation and the small Z channel 102 securely couple the medium sized nerve in the chamber 101.

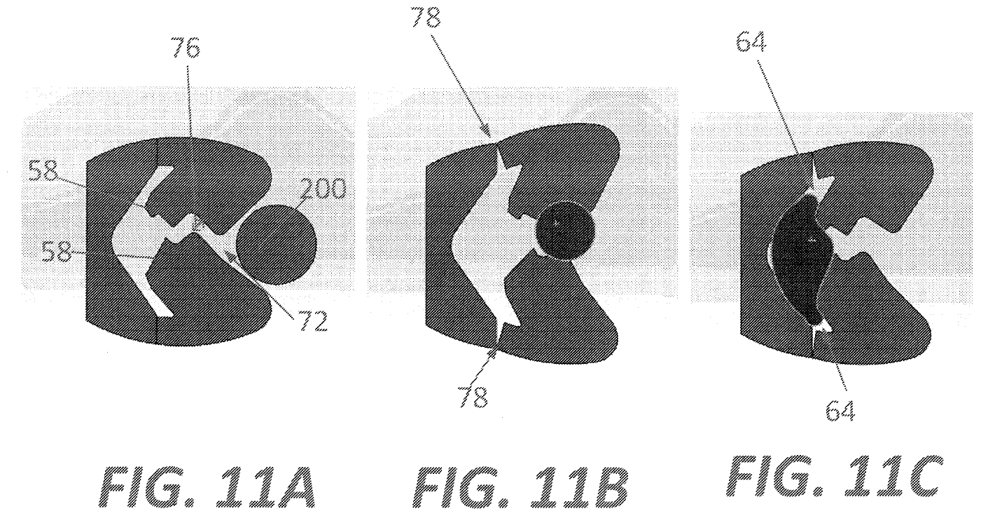

FIGS. 11A-11C schematically show a process of positioning a large nerve 200 (e.g., about 2.5 mm to about 3.5 mm in diameter) within the chamber 101 for stimulation. The figures show a side view of the device 100.

For the sake of discussion, the nerve 200 shown in FIGS. 11A-11C is described as a large nerve of about 3 mm in diameter. The nerve 200 may be positioned at the proximal end 72 of the channel as described previously.

As shown in FIG. 11B the nerve 200 may be stretched (e.g., by the surgeon) and/or squeezed (e.g., by the inner surface of the channel 102) to reduce the cross-sectional dimension of the nerve 200. Preferably, the reduction in nerve 200 dimension is less than 50% to assure safety to the nerve 200, as some damage might otherwise occur from greater reductions in cross-sectional size. For example, the nerve may be reduced to a cross-sectional dimension of about 2.25 mm. Assuming the channel dimension 76 is about 0.5 mm, the nerve 200 may still be too large to pass through the channel 102. To that end, the hinges 78 open the jaws 50 and expand the channel dimension 76 to allow the large nerve 200 to pass through the channel.

As shown in FIG. 11C, when the nerve 200 passes the distal end 74 of the channel 102 and enters the chamber 101, the jaws 50 may again close. However, because of the large size of the nerve 200, the jaws 50 may or may not be able to close entirely so as to return the channel 102 to its original dimension 77. Regardless, the jaws 50 retain the large nerve 200 within the chamber 101. To that end, the jaws 50 may include deformation areas 64 where the nerve 200 shape may deform and expand when compressed. Additionally, to assist with reducing nerve 200 compression, the nerve contact surfaces 58 may be configured to deform, thereby giving the nerve 200 additional room to expand within the chamber 101. Furthermore, in some embodiments, and as shown in FIG. 11C, the channel 102 dimension 76 may be expanded in the resting configuration because of interference with the large nerve 200.

Because of the reduced channel dimension 76 and the non-linear channel 102 shape, the small nerve 200 is securely coupled within the chamber 101. Although not drawn to scale, the jaws 50 may include nerve contact surfaces 58 configured to press and/or hold the nerve 200 against the electrode 104.

It should be understood that various embodiments advantageously allow the nerve 200 to expand/deform within the chamber 101, as opposed to a compensatory expansion/deformation that might otherwise occur outside of the chamber 101 and damage the nerve 200. However, in various embodiments, some deformation outside of the chamber 101 may occur. Preferred embodiments advantageously provide jaws with deformable nerve contact portions, as well as designated deformation areas within the device to allow for deformation adjacent/local to the area of compression on the nerve. As is known in the art, nerves are made up of bundles of axons. By allowing localized deformation of the nerve, the position of individual axons and/or fascicles may be rearranged without necessarily damaging any individual axon (as might occur if the nerve were to drastically compress or stretch.

Stress incontinence, overactive bladder, and fecal incontinence are undesirable conditions experienced by patients around the world. Stress incontinence happens when physical movement or activity—such as coughing, laughing, sneezing, running or heavy lifting—puts pressure (stress) on the bladder, causing it to leak urine. Overactive bladder, on the other hand, causes a frequent and sudden urge to urinate that may be difficult to control. Patients feel like they need to pass urine many times during the day and night, and may also experience unintentional loss of urine (urgency incontinence).

Through experimentation, the inventors discovered that both OAB and SUI may be treated by stimulating the same nerve with precise control signals. Furthermore, the inventors believe that both OAB and FI may be treated by stimulating the same nerve, also with precise control signals. However, each condition may be treated using different stimulation parameters. In particular, the inventors have determined that the neuromodulation device 100 may provide an SUI control signal using prescribed SUI control parameters to treat or improve symptoms of SUI. Additionally, or alternatively, the device 100 may provide an OAB control signal using prescribed OAB control parameters to treat or improve symptoms of OAB. Additionally, or alternatively, the device 100 may provide a FI control signal using prescribed FI control parameters to treat or improve symptoms of FI. Furthermore, the inventors have determined that these control signals may be provided in specific treatment regiments to result in short-term and/or long-term treatment of the aforementioned conditions or symptoms thereof.

Generally, electrical stimulation conductively travels both afferently and efferently from the point of stimulation (e.g., the perineal nerve) along conductive biological pathways. However, the inventors discovered that a first set of stimulation parameters provide a control signal effectively useful for OAB conducted afferently, but not necessarily clinically useful for SUI. In a similar manner, the inventors discovered that a second set of stimulation parameters provide a control signal effectively useful for SUI when conducted efferently, but not necessarily clinically useful for OAB. Furthermore, illustrative embodiments provide another set of stimulation parameters of a control signal effectively useful for FI conducted afferently, but not necessarily clinically useful for SUI. Similarly, illustrative embodiments provide another set of stimulation parameters of a control signal effectively useful for FI conducted efferently, but not necessarily clinically useful for OAB. In other words, one control signal provides a first clinical benefit, a second control signal provides a second, different clinical benefit, but neither of these signals necessarily is useful for both. This enables a targeted treatment from a single node (i.e., single nerve) for either OAB, FI and/or SUI. In some embodiments, two control signals can be modulated or interleaved to provide both benefits.

The beneficial stimulation control parameters are discussed below relative to treatment of various physiological conditions (e.g., OAB, SUI, and FI). Thus, even though all stimulation signals travel both afferently and efferently, only specific stimulation parameters are configured to cause a control effect related to the physiological condition in a particular direction (treatment of a particular symptom of a condition afferently or efferently), and those signals are referred to as control signals.

For example, the first set of stimulation parameters cause the stimulation signal to become an afferent OAB control signal (i.e., from the stimulated nerve to the CNS), and the second set of stimulation parameters cause the stimulation signal to become an efferent SUI control signal to (i.e., from the stimulated nerve to the muscle/sphincter).

Accordingly, for OAB, the afferent portion of the specially configured control signal is clinically useful— the efferent portion of that control signal does not necessarily have beneficial clinical effect on OAB. Therefore, for simplicity, this OAB control signal also may be referred to as an “afferent control signal.” In a similar manner, for SUI, the efferent portion of the specially configured control signal is clinically useful—the afferent portion of that control signal does not necessarily have the beneficial clinical effect on OAB. Therefore, for simplicity, this SUI control signal also may be referred to as an “efferent control signal.”

In addition, the term “control signal” may also be referred to as a “treatment signal.” A first set of afferent parameters and a second set of efferent parameters (referred to for convenience as “afferent” and “efferent”) differ in various ways, as discussed further below. Accordingly, illustrative embodiments can be said to provide an SUI control signal, FI control signal, and/or an OAB control signal, without being limited to any particular mechanism of action. Furthermore, although various embodiments describe treatment of one or more conditions using an afferent control signal and efferent control signal, it should be understood that some embodiments may use only a single type of control signal (e.g., either afferent or efferent signal). In various embodiments, the afferent control signal may treat a sensory type symptom of the patient (e.g., patient feels the urge to urinate), whereas the efferent control signal may treat a motor related symptom of the patient (e.g., sphincter applies insufficient pressure on bladder).

As known by those skilled in the art, nerves can be categorized as motor nerves or sensory nerves because of the predominance of fibers that the nerve contains. However, almost all nerves contain a mix of afferent (sensory) fibers, efferent (motor) fibers, and autonomic fibers. Illustrative embodiments stimulate a motor nerve using the neuromodulation device 100 to activate afferent pathways to treat OAB and efferent pathways to treat SUI.

TABLE 1
TREATMENT PARAMETERS FOR OAB, FI, AND SUI IN HUMANS

| | Target | | Amplitude | | Duty | Treatment |
|---|---|---|---|---|---|---|
| | Nerve | Frequency | of the pulse | Pulse Duration | Cycle | Duration |
| OAB | Perineal | 2 Hz-20 Hz | Sub- | 200 microsec.- | (a) 15 min | About 10 |
| (afferent | Nerve | | threshold. | 300 microsec. | continuous | min.- |
| control | | | Monophasic | | on to | about 30 |
| signal) | | | or Bi-Phasic | | (b) 15 sec | min. |
| and/or | | | pulse. | | ON, 2.5 | 1-3× a |
| FI | | | 0.4 mA to | | min OFF, | day. |
| (afferent | | | 1.0 mA. | | repeating | |
| control | | | | | 4 times. | |
| signal) | | | | | | |
| SUI | Perineal | 50 Hz-80 Hz | Threshold | 250 microsec.- | 15 sec ON, | About 10 |
| (efferent | Nerve | | Bi-Phasic | 400 microsec. | 2.5 min | min.- |
| control | | | pulse. | | OFF, | about 13 |
| signal) | | | 0.5 mA to | | repeating | min. |
| | | | 2.0 mA | | 4 times. | 1-3× a |
| | | | | | | day. |

In various embodiments, the controller 107 is configured to set the above stimulation parameters and to relay a signal to cause the electrodes to stimulate in accordance with the above parameters. Although the above parameters describe optimal stimulation parameters, illustrative embodiments may use stimulation parameters beyond what is described above for various nerves. For example, the controller 107 is configured to set a duty cycle for a treatment duration, where the duty cycle is a ratio of the active duration to the period.

In various embodiments, the duty cycle for OAB and/or SUI treatment is between about 15:150 (active:inactive), also referred to as a 10% duty cycle. In some embodiments, the duty cycle for OAB and/or SUI treatment may be 30:150, also referred to as a 20% duty cycle. In various embodiments, the duty cycle may be between 5% and 25%. However, as shown above, in some embodiments the duty cycle may be about 100% (continuous or near continuous stimulation as opposed to a plurality of smaller pulses). Thus, various embodiments may have a duty cycle of between about 5% and 100%. In general, the higher the frequency, the lower the duty cycle and vice-versa (for OAB, SUI, and/or FI treatment). For example, illustrative embodiments may provide a 100% duty cycle with a 2 Hz frequency for the OAB or FI control signal. Some other embodiments may provide a 10% duty cycle with a 20 Hz frequency for the OAB or FI control signal.

In various embodiments, the stimulation pulse may be monophasic or biphasic. In general, biphasic stimulation signals are preferred for patient safety. It is theorized that by reversing the pulse via biphasic stimulation, the cells more closely return to the normal physiological stage. However, some embodiments may use monophasic pulses, particularly at lower frequencies (e.g., for OAB treatment). Monophasic pulses can present more of a safety issue at higher frequencies because higher frequency puts more strain on the body. Thus, in various embodiments the higher-frequency efferent treatment may use bi-phasic stimulation. For lower frequency afferent stimulation, the body has more time to return to normal basal levels and is therefore better able to deal with monophasic stimulation.

Various embodiments may use sub-threshold stimulation. As known in the art, stimulation over a certain intensity level elicits a (response (e.g., motor or pressure response). For OAB control signal, threshold stimulation causes the movement of the muscle (e.g., closing of the urethra, increase of bladder pressure). For efferent control signals, sub-threshold stimulation provides little or no muscle twitch. Sub-threshold activation intensity (i.e., amplitude) for efferent stimulation signals may cause the innervated muscle to show a muscle contraction response that is 50% or less of the maximum response of the target muscle movement. In various embodiments, sub-threshold intensity results in no motor response for the target muscle. For afferent control signals, sub-threshold stimulation is the signal intensity (i.e., amplitude) that is 10% or less of the intensity that invokes a minimally observable response.

Although treatment for both OAB and SUI may occur using the same stimulation device 100 by stimulating the same nerve, the stimulation parameters for treatment of OAB and SUI are markedly different. The OAB control stimulation parameters generally require a significantly lower frequency that the SUI control stimulation parameters. In particular, illustrative embodiments use subthreshold OAB control signals to treat OAB (i.e., amplitude does not cause an action potential in the nerve/does not elicit a motor response). Various embodiments may determine a sub-threshold amplitude by stimulating the nerve until the muscle contracts, and then lowering the amplitude until the muscle no longer contracts. Accordingly, the sensory fibers may be stimulated, but not the motor fibers. The inventors determined that sub-threshold stimulation provides a signal through the patients afferent pathways that assists with the response of quieting the bladder. Accordingly, such OAB stimulation parameters may be used for treatment of OAB and urge incontinence, and/or mixed incontinence.

Although illustrative embodiments refer to stimulating the perineal nerve, it should be understood that a number of other nerves may also be stimulated to achieve treatment for OAB and SUI. These nerves include the tibial nerve, or another pelvic nerve, such as, clitoralis, inferior rectal, and/or any branches of the prudendal and levator ani nerves, pubococygeus, also known as pubovisceral, Iliococcygeus, Puboperineal, Pubovaginal, Puboanal, Puborectal nerves.

The inventors have obtained data in a variety of animals indicating that efferent control signals assist in closure of the urinary sphincter for individuals have SUI.

Through experimentation, the inventors discovered that motor nerves can be stimulated for positive treatment effects with unique afferent and efferent control signals.

Thus, direct stimulation of pelvic floor nerves can activate a type of sensory fiber not previously associated with overactive urinary incontinence. Specifically, this includes the proprioceptive type Ia and Ib large myelinated sensory afferents. Using the appropriate parameters/specifications, these sensory afferents can then be targeted for selective neuromodulation to activate a previously unknown sensory pathway to the central nervous system, including pathways in the spinal cord and brainstem involved in storage/voiding control.

The inventors were further surprised to learn that acute wireless electrical stimulation of the bulbospongiosus nerve in certain animals (e.g., rabbits) increased the maximal urethral pressure and voiding efficiency significantly, proportional to the stimulation frequency. Acute stimulation was sufficient to induce a 3-fold increase in urethral pressure and voiding volume, indicating that activation of this perineal nerve strengthens the urethral sphincter supporting continence and voiding efficiency. This result confirmed the ability of miniature wireless stimulators for effective pelvic floor muscle contraction, and as a strategy to improve and/or reverse SUI-like features in aging and multiparous rabbits.

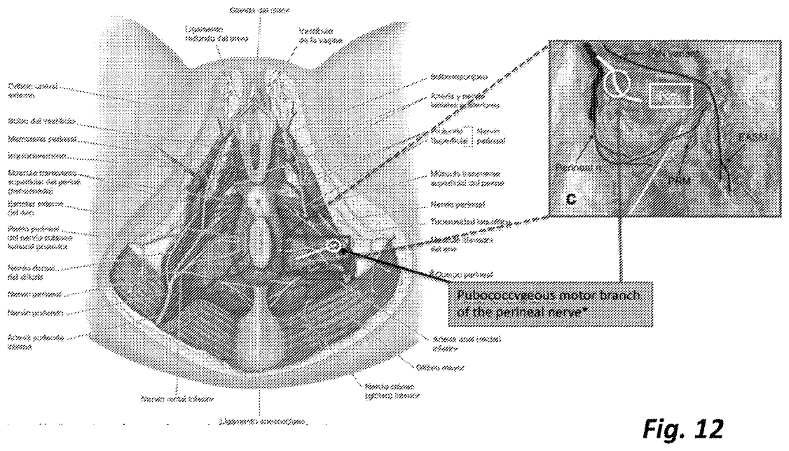

FIG. 12 is an anatomical drawing showing various nerves and anatomy relevant to illustrative embodiments. The coccygeal nerve plexus is anatomically and functionally different compared to the pelvic nerve plexus, which includes the pudendal nerve. Therefore, sacral neuromodulation (SNM), percutaneous tibial nerve stimulation (PTNS), and pudendal nerve stimulation (PNS), all part of the pelvic plexus, target the same sacral (S2-3) levels in the spinal cord and are mixed anatomically and functionally with pain, mechanoception sensory neurons, mixed with motor proprioceptive and autonomic neurons. The mechanism of action preferably involves the modulation of the mechanosensory afferent fibers.

In contrast, the inventors noted that the coccygeal plexus is more uniform anatomically and functionally, and composed mostly from motor somatic efferent and proprioceptive (Ia/Ib) sensory afferents, which enter the spinal cord through a more posterior (S4-5) spinal cord levels. Yet, the proprioceptive axons ascend all the way to L7 connecting the function of the PFMs to those of the bladder and other pelvic organs. The inventors thus discovered that stimulation of the proprioceptive sensory afferents also inhibits the activity from the bladder and thus, discovered that such stimulation can be a useful treatment for overactive bladder syndrome. Illustrative embodiments have selected the motor branch of the perineal nerve, which controls the pubococcygeous muscle.

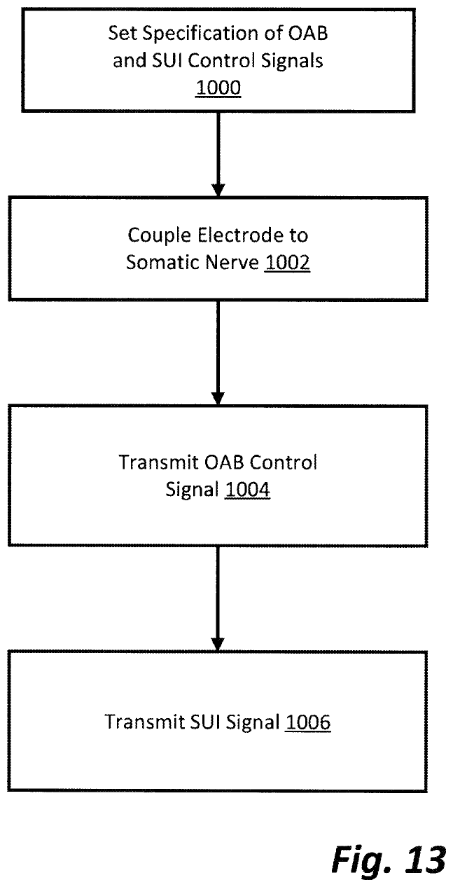

FIG. 13 shows a neuromodulation process in accordance with illustrative embodiments. It should be noted that this process is simplified from a longer process that normally would be used to neuromodulate a nerve. Accordingly, the process may have many other steps that those skilled in the art likely would use. In addition, some of the steps may be performed in a different order than that shown, or at the same time. Those skilled in the art therefore can modify the process as appropriate. Moreover, as noted above and below, materials, devices, and structures discussed by in this description are exemplary—those skilled in the art can select the appropriate materials and structures depending upon the application and other constraints.

The process uses at least one “control signal” to neuromodulate the appropriate coccygeal plexus somatic nerve, which, in this example, is the perineal nerve. Those control signals are referred to as:
1) The “OAB control signal”—stimulation parameters directed toward managing overactive bladder syndrome, and
2) The “SUI control signal”—stimulation parameters directed toward managing stress urinary incontinence.

The process of FIG. 13 begins at step 1000, which sets the specifications of the OAB and SUI signals. These specifications may be stored in a database, memory, or other storage medium for use in subsequent steps. Among other locations, such a location may be part of the device 100 itself, part of the signal generator, some somewhere across a local area network (e.g., an enterprise network), or somewhere across a larger wide area network (e.g., the Internet). The inventors determined, using calculations and experimentation, that the succeeding specifications should produce satisfactory results.

Specifically, the OAB control signal may have a set of one or more of the following specifications for humans:
an amplitude of between about 0.4 milliamps and 1 milliamp,
each pulse having a pulse duration of between about 200 microseconds and 400 microseconds (when the OAB control signal is a periodic signal),
a frequency of between about 5 Hertz and 20 Hertz (when the OAB control signal is a periodic signal), and
duty cycle of between about 5% and 100% (e.g., more specifically between about 8% and 12%).
a stimulation pulse of about 10-20 seconds followed by about 2.5 minutes off, repeated at least 3-4 times within a single session
a duration for transmitting the OAB control signal of no less than 10 minutes and for no longer than 30 minutes in a single session.

These values can be scaled appropriately for different mammals. In addition, these values can be adjusted as a function of the number of treatments per day or week, the individual, and/or the disease severity.

The SUI control signal may have a set of one or more of the following specifications for humans:
an amplitude of between about 0.5 milliamps and 2 milliamp,
each pulse having a pulse duration of between about 200 microseconds and 400 microseconds (e.g., when the OAB control signal is a periodic signal),
a frequency of between about 60 Hertz and 100 Hertz (when the OAB control signal is a periodic signal), and
duty cycle of between about 5% and 100% (e.g., more specifically between about 8% and 12%).
a stimulation pulse of about 10-20 seconds followed by about 2.5 minutes off, repeated at least 3-4 times within a single session
a duration for transmitting the SUI control signal of no less than 30 seconds and for no longer than 120 seconds in a single session.

As with the specifications for the OAB control signal, these values can be scaled appropriately for different mammals. In addition, these values can be adjusted as a function of the number of treatments per day or week, the individual, and/or the disease severity.

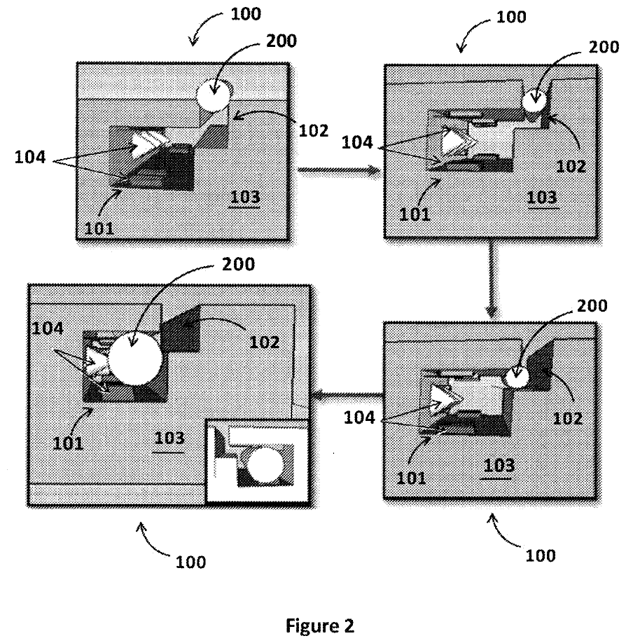

The process continues to step 1002, which couples the electrode 104 to the somatic nerve 200 (e.g., the perineal nerve) as discussed above. Accordingly, a physician may stretch and slide the perineal nerve 200 and secure it within the chamber 101 as shown, for example in FIG. 2. When coupled in the chamber 101, depending on its size, the nerve 200 may relax or return toward its normal state such that the nerve 200 is captured within the chamber 101.

As noted above, the nerve preferably is directly and conductively coupled with the electrode 104. Accordingly, there preferably is no other non-negligible organic component of the patient between the nerve and the device 100 (e.g., the electrode 104 and the nerve). Some embodiments may have a coating or other structure on the electrode 104 and still have a direct connection or coupling with the nerve 200.

With the nerve 200 securely in the chamber 101, the signal generator may begin transmitting the OAB control signal to the device 100, through the electrode 104, and to the perineal nerve (step 1004). This activates the pelvic floor in a prescribed manner. During experimentation, however, the inventors were surprised to discover that this signal seemed to mitigate the effect of the natural OAB signal on the pelvic floor. Specifically, the patient's body naturally sends an OAB signal (referred to as the “natural OAB signal”), which is a part of the patient's mechanism that triggers symptoms of OAB. This activation on the perineal nerve, however, seems to block or otherwise interfere with the body's reaction to the natural OAB signal, blunting or otherwise mitigating the impact of the natural OAB signal. Furthermore, the inventors determined that treatment resulted in long-lasting effects that extended beyond the immediate stimulation period.

Illustrative embodiments may improve OAB symptoms for a plurality of days (e.g., 7 days) after a single treatment. Furthermore, illustrative embodiments may improve OAB symptoms for a plurality of months (e.g., 4-6 months) after 12 days of treatment. In some embodiments, the SUI symptoms may be improved after about 5-10 days of treatments. In some embodiments, SUI symptoms may be improved long-term after ceasing stimulation. Accordingly, stimulation in accordance with illustrative embodiments described herein improves and/or normalizes the OAB symptoms. In various embodiments, the patient may be treated for as long as the underlying cause of OAB is present. In some embodiments, the neuromodulate device 100 is intended as a long term or permanent implant that is bounded only by batter life. As battery tech improves, various embodiments may implant the device 100 for the life of the user.

In some embodiments, the OAB control signal is also an afferent FI control signal. The control signals have the same parameters. Furthermore, the inventors determined that afferent stimulation of the perineal nerve (provided by the OAB control signal) also improves symptoms of Fecal Incontinence. Accordingly, in some embodiments, the OAB control signal of step 1004 also operates as an afferent FI control signal.

As noted above, for a single session, the method may transmit the OAB control signal to the perineal nerve for between about 10 and 30 minutes. As noted, this timing can be fine-tuned as a function of the patient, severity of the disease, etc. After the OAB signal treatment is complete, then the process may continue to optional step 1006 to also treat SUI. Specifically, if the patient also suffers from SUI, then the method may transmit the SUI control signal to the perineal nerve, in this session, for between 30 and 120 seconds. Other embodiments may swap the order of steps 1004 and 1006. Furthermore, in some embodiments, steps 1004 and 1006 may occur immediately after one another, minutes apart, hours apart, or days apart. For example, illustrative embodiments may provide OAB treatment signals for a treatment period of about 30 minutes, wait 1 hour, provide SUI treatment signals for a treatment period of about 15 minutes, wait 1 hour, and then repeat the process. The inventors determined that using this method of treatment, that the OAB treatment is likely to be repeated more often per day (e.g., 2-3× per day) for OAB daily, while SUI may be treated less often (e.g., 1× per day). Thus, in some embodiments, the OAB treatment may occur many times in a day, but the SUI treatment may be a single daily treatment. The process then comes to an end.

Accordingly, the inventors discovered that they could treat both OAB and SUI via a relatively easy to access somatic nerve (e.g., the perineal nerve). Importantly, this nerve is quite small, thus requiring a much lower power than required for many other conventional neurostimulation systems.

It should be apparent that the above described process advantageously requires only a single surgical intervention for the treatment of two conditions OAB and SUI from the same neuromodulation device 100 coupled with the same nerve 200 (i.e., by using afferent control signals for OAB and efferent control signals for SUI). Furthermore, it should be apparent that various embodiments may skip either of steps 1004 or 1006, i.e., some embodiments may treat one, but not both, of OAB and SUI.

The inventors also determined that illustrative embodiments may treat fecal incontinence, in a manner similar to the treatment of OAB and SUI described above. Fecal incontinence (FI) is a challenging condition defined as a recurrent, uncontrolled passage of fecal material for at least 1 month in an individual older than 4 years of age. This condition affects as many as 18% of people based on community surveys, with rates rising to 55% in the elderly. The etiology of FI includes altered stool consistency, abnormal rectal capacity, decreased anorectal sensation, and pelvic floor or anal sphincter dysfunction. The morphological change of the anorectal junction, structural anal sphincter damage, preoperative radiotherapy, and neorectal dysfunction are likely causes of fecal incontinence.

The external anal sphincter (EAS) is a striated muscle that envelops rectum. The deepest part of the EAS is intimately related to the puborectalis muscle (PRM), which is part of both the levator ani and EAS muscle complexes. The anal sphincter consists of the internal anal sphincter, which is a 0.3-0.5 cm thick expansion of the circular smooth muscle layer of the rectum, and the external anal sphincter which is a 0.6-1 cm thick expansion of the striated levator ani muscles. Morphologically, both sphincters are separate and heterogeneous During defecation, the puborectalis muscle relaxes, the angle straightens, and the stool can be pushed downward to and through the anal canal. The neuromuscular integrity of the rectum, anus, and the adjoining pelvic floor musculature helps to maintain normal fecal continence. The EAS is by branches of the pudendal nerves, while the PRM is innervated by a branch of the levator ani nerve.

Anatomic disruption of the EAS or PRM to obstetric trauma is the most common surgically correctable cause of fecal incontinence, affecting patients are often relatively young with major symptoms of incontinence. In denervation of the PRM and EAS muscles has been described in up to 80% of patients with idiopathic fecal incontinence. Direct trauma to the anal sphincter or associated nerves during vaginal delivery are common causative factors contributing to FI. Up to 35% of women may experience injuries to the anal sphincter following vaginal delivery, even without visible signs of perineal injury. Pregnancy, vaginal delivery, parity, smoking, and body mass index are risk factors for PFD, and its prevalence increases with age. Maximal urethral and anal closure pressure, depend on pelvic floor muscle (PFM) health, and disturbances in pelvic floor nerves or muscles have strong association with Urinary and/or Fecal incontinence. In fact, up to 35% of women experience injuries to the anal sphincter following vaginal delivery, even without visible signs of perineal injury. Specific PFM are involved in normal function of urinary and fecal retention working as sphincters, when affected, are unable to achieve normal muscle contraction when autonomically or consciously desired, causing or contributing to incontinence.

Illustrative embodiments use the neuromodulation device 100 to stimulate particular nerves, both using an afferent control signal and an efferent control signal, to assist with reduction and treatment of FI symptoms.

Some embodiments may use sacral nerve stimulation. For sacral nerve stimulation, the principal nerve that is stimulated is the pudendal nerve. Stimulation of the pudendal nerve increases anal pressure, whereas stimulation of S3 increases pressure only slightly, but causes an impressive decrease of the rectoanal angle. Increases in anal pressure indicate that the subject is able to hold their stool for longer and/or hold more stool. A weak sphincter will produce less pressure and thus contribute to fecal incontinence. When S3 was stimulated after bilateral pudendal block, anal pressure did not change, but the decrease in the rectoanal angulation persisted. The changes in anal pressure could be obtained without fatigue at stimulation frequencies of 10 to 20 Hz. Sacral nerve stimulation therapy has been shown to decrease weekly episodes of fecal incontinence from a mean of 9.1 at baseline to 1.7 at 5 years; 89% of patients had a greater than 50% improvement.

However, sacral nerve stimulation requires a two-step surgical implantation procedure in order to select for responders (approximately 47%) and determine a permanent position for the lead electrode. This is needed since sacral stimulators use lead wires, which are placed near the target nerves and might move after placement. Sacral stimulators use an implantable pulse generator connected to a lead electrode placed at the at the S3-S4 level and intended to activate afferent pathways associated with the sense of fecal urge. These devices rely on volume conduction which indiscriminately activate the nerves controlling several organs including urethra, external anal sphincter, levator ani muscles, perineal skin and clitoris. Estimated risk of removal at median follow-up of 2 years was 11.8%. Illustrative embodiments preferably provide a single surgical procedure for installation of the neuromodulation device 100 for treatment of FI.

Illustrative embodiments discovered that efferent stimulation of the inferior rectal nerve (a branch of the pudendal nerve) efficiently contracts the rectal sphincter, and thus, can be used as a target for neuromodulation treatment for FI. The inferior rectal nerve can be targeted for selective neuromodulation both directly.

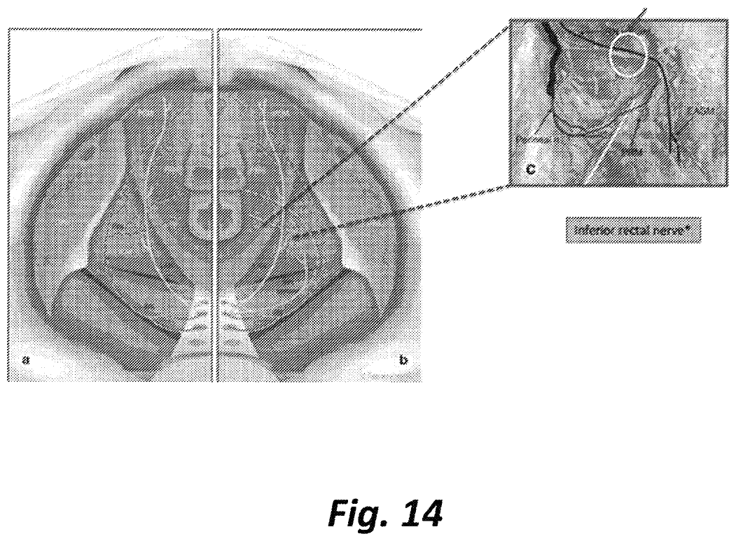

FIG. 14 is an anatomical drawing showing various nerves and anatomy relevant to illustrative embodiments. The coccygeal plexus is anatomically and functionally different as compared to the pelvic plexus, which includes the pudendal nerve. Therefore, SNM, PTNS, and PNS, which all part of the pelvic plexus, target the same sacral (S2-3) levels in the spinal cord. These are mixed anatomically and functionally, and their main proposed mechanism of action being the modulation of the sensory afferent fibers is similar.

In sharp contrast, the inventors determined that the coccygeal plexus is more uniform anatomically and functionally, and composed mostly from motor in somatic efferent and proprioceptive (Ia/Ib) sensory afferents, which enter the spinal cord through a more posterior (S4-5) spinal cord levels. Yet, the proprioceptive axons ascend all the way to L7 connecting the function of the PFMs to those of the bladder and other pelvic organs.

The external anal sphincter receives both motor and sensory innervation from the inferior rectal nerve, a branch of the pudendal nerve. Although illustrative embodiments may be used to stimulate almost any nerve in the pelvic area, various embodiments stimulate the inferior rectal nerve to activate the external urinary sphincter (EUS) and the puborectalis muscle (PRM), which supports the closure of the rectal sphincter.

Variants of the inferior rectal nerve have been documented, wherein the nerve arises independently from the sacral plexus, with independent derivation from the fourth sacral root (S4), and more distally, piercing of the sacrospinous ligament. Other variants include the inferior rectal nerve arising from S4 and passing posteriorly to the sacrospinous ligament; the inferior rectal nerve arising from S4 has also been observed to rejoin the pudendal nerve before passing the sacrospinous ligament, and arising intrapelvically from the S3 ventral ramus, then distally gives off a branch that joins the continuation of the pudendal nerve in the perineum. Stimulation of the distal segment of the inferior rectal nerve (IRN) advantageously obviates such variability as it is closer to the EAS and PRM targets.

In a process similar to that described above for treating SUI and OAB, FI can be treated using an afferent and/or efferent stimulation signal. Illustrative embodiments present a novel treatment of FI using efferent control signals. The inventors suspect that treatment of FI using both efferent and afferent control signals will lead to enhanced treatment outcomes. Advantageously, both afferent and efferent control signals may be transmitted on the same nerve using the same neuromodulation device 100.

TABLE 2
TREATMENT PARAMETERS FOR FI AND OAB IN HUMANS

| | Target | | | | Duty | Treatment |
|---|---|---|---|---|---|---|
| | Nerve | Frequency | Amplitude | Pulse Duration | Cycle | Duration |
| FI (afferent | Inferior | 2 Hz-20 Hz | Sub- | 200 microsec.- | (a) 15 | About 10 |
| control | Rectal | | threshold. | 300 microsec. | min | min.- |
| signal) | Nerve | | Bi-Phasic | | continuous | about 30 |
| and/or | | | pulse. | | to | min. |
| OAB | | | 0.4 mA to | | (b) 15 | 1-3× a day. |
| (afferent | | | 1.0 mA. | | sec | |
| control | | | | | ON, 2.5 | |
| signal | | | | | min | |
| | | | | | OFF, | |
| | | | | | repeating | |
| | | | | | 4 times | |
| FI | Inferior | 50 Hz-80 Hz | Threshold | 250 microsec.- | 15 sec | About 10 |
| (efferent | Rectal | | Bi-Phasic | 400 microsec. | ON, 2.5 | min.- |
| control | Nerve | | pulse. 0.5 | | min | about 13 |
| signal) | | | mA to 2.0 | | OFF, | min. |
| | | | mA | | repeating | 1-3× a day. |
| | | | | | 4 times. | |

Experimentation by the inventors has shown that efferent stimulation of the inferior rectal nerve using the above referenced stimulation parameters may close the external, and secondary rectal sphincter.

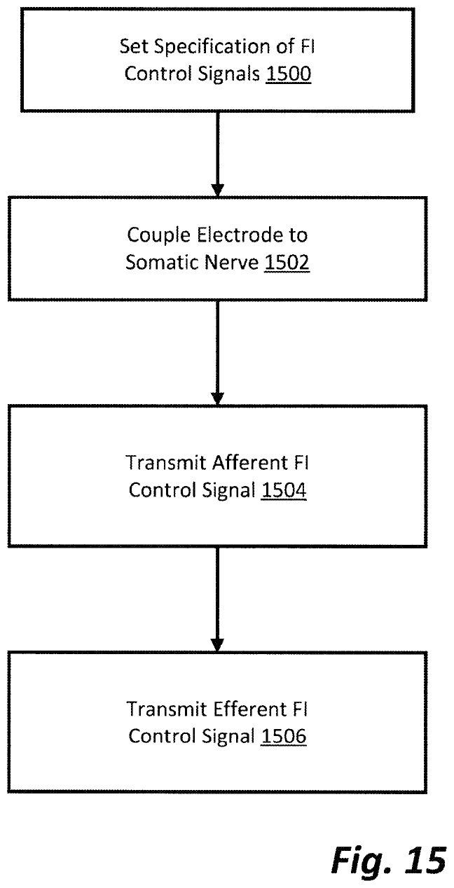

FIG. 15 shows a neuromodulation process in accordance with illustrative embodiments. It should be noted that this process is simplified from a longer process that normally would be used to neuromodulate a nerve. Accordingly, the process may have many other steps that those skilled in the art likely would use. In addition, some of the steps may be performed in a different order than that shown, or at the same time. Those skilled in the art therefore can modify the process as appropriate. Moreover, as noted above and below, materials, devices, and structures discussed by in this description are exemplary—those skilled in the art can select the appropriate materials and structures depending upon the application and other constraints.

The process uses at least one “control signal” to neuromodulate the appropriate coccygeal plexus somatic nerve, which, in this example, is the perineal nerve. Those control signals are referred to as:
1) The afferent control signal—configured to stimulate the motor fibers of the nerve, and
2) The efferent control signal—configured to stimulate the sensory fibers of the nerve,

The process of FIG. 15 begins at step 1500, which sets the specifications of the afferent and efferent control signals. These specifications may be stored in a database, memory, or other storage medium for use in subsequent steps. Among other locations, such a location may be part of the device 100 itself, part of the signal generator, some somewhere across a local area network (e.g., an enterprise network), or somewhere across a larger wide area network (e.g., the Internet). The inventors determined, using calculations and experimentation, that the succeeding specifications should produce satisfactory results.

Specifically, the afferent control signal (e.g., afferent FI control signal) may have a set of one or more of the following specifications for humans:
an amplitude of between about 0.4 milliamps and 1 milliamp,
each pulse having a pulse duration of between about 200 microseconds and 300 microseconds (when the afferent control signal is a periodic signal),
a frequency of between about 5 Hertz and 20 Hertz (when the afferent control signal is a periodic signal), and
duty cycle of between about 5% and 100% (e.g., more specifically between about 8% and 12%).
a stimulation pulse of about 10-20 seconds followed by about 2.5 minutes off, repeated at least 3-4 times within a single session
a duration for transmitting the afferent control signal of no less than 10 minutes and for no longer than 30 minutes in a single session.

These values can be scaled appropriately for different mammals. In addition, these values can be adjusted as a function of the number of treatments per day or week, the individual, and/or the disease severity.

The efferent control signal (e.g., efferent FI control signal) may have a set of one or more of the following specifications for humans:
an amplitude of between about 0.5 milliamps and 2 milliamp,
each pulse having a pulse duration of between about 200 microseconds and 400 microseconds (when the OAB control signal is a periodic signal),
a frequency of between about 60 Hertz and 100 Hertz (when the OAB control signal is a periodic signal), and
duty cycle of between about 5% and 100% (e.g., more specifically between about 8% and 12%).
a stimulation pulse of about 10-20 seconds followed by about 2.5 minutes off, repeated at least 3-4 times within a single session
a duration for transmitting the SUI control signal of no less than 30 seconds and for no longer than 120 seconds in a single session.

As with the specifications for the afferent control signal, these values can be scaled appropriately for different mammals. In addition, these values can be adjusted as a function of the number of treatments per day or week, the individual, and/or the disease severity.

The process continues to step 1502, which couples the electrode 104 to the somatic nerve 200 (e.g., the inferior rectal nerve) as discussed above. Accordingly, a physician may stretch and slide the inferior rectal nerve 200 and secure it within the chamber 101 as shown, for example in FIG. 2. When coupled in the chamber 101, depending on its size, the nerve 200 may relax or return toward its normal state such that the nerve 200 is captured within the chamber 101.

As noted above, the nerve preferably is directly and conductively coupled with the electrode 104. Accordingly, there preferably is no other non-negligible organic component of the patient between the nerve and the device 100 (e.g., the electrode 104 and the nerve). Some embodiments may have a coating or other structure on the electrode 104 and still have a direct connection or coupling with the nerve 200.

With the nerve 200 securely in the chamber 101, the signal generator may begin transmitting the afferent control signal to the device 100, through the electrode 104, and to the inferior rectal nerve (step 1504). This activates the pelvic floor in a prescribed manner. During experimentation, however, the inventors were surprised to discover that this signal seemed to mitigate the effect of the natural FI signal on the pelvic floor. Specifically, the patient's body naturally sends an FI signal (referred to as the “natural FI signal”), which is a part of the patient's mechanism that triggers some symptoms of FI. This activation on the inferior rectal nerve, however, seems to block or otherwise interfere with the body's reaction to the natural FI signal, blunting or otherwise mitigating the impact of the natural FI signal. In various embodiments, FI treatment results in long-lasting effects that extended beyond the stimulation period (e.g., at least one week, preferably at least 30 days). Accordingly, stimulation in accordance with illustrative embodiments described herein improves and/or normalizes the FI symptoms, at least temporarily. In some embodiments, the FI symptoms may be improved after about 5-10 days of treatments.

In some embodiments, the afferent FI control signal is also an OAB control signal. The afferent FI and OAB control signals have the same parameters. Furthermore, the inventors determined that afferent stimulation of the inferior rectal nerve (provided by the afferent FI control signal) also improves symptoms of OAB. Accordingly, in some embodiments, the afferent FI control signal of step 1504 also operates as an OAB control signal.

As noted above, for a single session, the method may transmit the afferent control signal to the perineal nerve for between about 10 and 30 minutes. In various embodiments, the afferent FI control signal may calm down the digestive tract for patients experiencing fecal incontinence. The efferent FI control signal may cause closure of the rectal sphincter. For SUI, the overactive bladder signals that mediate urgency are calmed down. As noted, this timing can be fine-tuned as a function of the patient, severity of the disease, etc. After the afferent signal treatment is complete, then the process may continue to optional step 1506 to further treat FI using the efferent control signal. Other embodiments may swap the order of steps 1504 and 1506. Furthermore, in some embodiments, steps 1504 and 1506 may occur immediately after one another, minutes apart, hours apart, or days apart. For example, illustrative embodiments may provide afferent treatment signals for a treatment period of about 30 minutes, wait 1 hour, provide efferent treatment signals for a treatment period of about 15 minutes, wait 1 hour, and then repeat the process. Some embodiments may skip either one of steps 1504 or 1506. The process then comes to an end.

Although illustrative embodiments have been described and providing treatment options by stimulating the perineal nerve or the inferior rectal nerve, some embodiments may use the same stimulation parameters to stimulate other nerves. For example, the following nerves may be stimulated using the OAB control signal to treat OAB, using the SUI control signal to treat SUI, using the afferent FI control signal to treat FT, and/or using the efferent FT control signal to treat FI: These nerves include the tibial nerve, or another pelvic nerve, such as, clitoralis, inferior rectal, and/or any branches of the pudendal, levator ani nerves and hypogastric nerve, including the pubococygeus, also known as pubovisceral, Iliococcygeus. Or from the Auerbach's nerve plexus, and lower rectal branches of the pelvic autonomic nerve plexu.

The device 100 and signal generator may be distributed separately, or together as a kit.

Some or all of this process may be implemented in hardware, software, or both. For example, some or all of this process may be implemented using a custom application specific integrated circuit, FPGA, microcontroller, or other logic, with software (e.g., firmware), and integrated with the rest of the system.

In various embodiments, the device 100 may include a controller 107 preconfigured to treat or improve one or more of the conditions described herein, namely OAB, FI, and/or SUI. In some embodiments, the controller 107 may be have, or communicate with, an internal or external storage database that includes one or more of the stimulation parameters described herein (e.g., in Table 1 or Table 2, among other places). A user, such as a medical practitioner, may communicate with and operate the controller 107 through a user interface. In various embodiments, the device 100 may contain a hard-wired or wireless (e.g., Bluetooth or internet) communication network. In various embodiments, the user interface may be a browser-based or smartphone application. Accordingly, illustrative embodiments may include a system having the controller 107 configured to stimulate the target nerve in accordance with the parameters described herein. The controller 107 may receive an indication from the medical practitioner regarding which nerve is being stimulated and/or which condition is being treated. The controller 107 may include the option of switching between threshold and sub-threshold simulation, and/or monophasic or biphasic simulation. Additionally, or alternatively, the controller 107 may allow for manual adjustments from a predefined template set of stimulation parameters. Furthermore, the controller 107 may include a trigger configured to initiate the stimulation session based on a pre-determined schedule, a user input, and/or establishment of a wired or wireless power connection.

Each of the above-described components (e.g., the controller, database, trigger, timer, etc.) may be operatively connected by any conventional interconnect mechanism, such as a bus communicating each of the components. Those skilled in the art should understand that this generalized description that can be modified to include other conventional direct or indirect connections. Accordingly, discussion of a bus is not intended to limit various embodiments.

Those skilled in the art should understand that each of these components can be implemented in a variety of conventional manners, such as by using hardware, software, or a combination of hardware and software, across one or more other functional components. For example, the controller 107 may be implemented using a plurality of microprocessors executing firmware. As another example, controller 107 may be implemented using one or more application specific integrated circuits (i.e., “ASICs”) and related software, or a combination of ASICs, discrete electronic components (e.g., transistors), and microprocessors. In some embodiments, the controller 107 may be distributed across a plurality of different machines—not necessarily within the same housing or chassis. Additionally, in some embodiments, components described as separate (such as the database and the controller 107) may be replaced by a single component. Furthermore, certain components and sub-components are optional. For example, some embodiments may not use the timer.

In some implementations, the processor includes one or more processors (or one or more processor cores) that each are configured to perform a series of instructions that result in manipulated data and/or control the operation of the other components of the controller 107. In some implementations, when executing a specific process (e.g., causing the electrode to output specific stimulation signals), the processor can be configured to make specific logic-based determinations based on input data received, and be further configured to provide one or more outputs that can be used to control or otherwise inform subsequent processing to be carried out by the processor and/or other processors or circuitry with which processor is communicatively coupled. Thus, the processor reacts to specific input stimulus in a specific way and generates a corresponding output based on that input stimulus. In some example cases, the processor can proceed through a sequence of logical transitions in which various internal register states and/or other bit cell states internal or external to the processor may be set to logic high or logic low. As referred to herein, the processor can be configured to execute a function where software is stored in a data store coupled to the processor, the software being configured to cause the processor to proceed through a sequence of various logic decisions that result in the function being executed. The various components that are described herein as being executable by the processor can be implemented in various forms of specialized hardware, software, or a combination thereof. For example, the processor can be a digital signal processor (DSP) such as a 24-bit DSP processor. The processor can be a multi-core processor, e.g., having two or more processing cores. The processor can be an Advanced RISC Machine (ARM) processor such as a 32-bit ARM processor. The processor can execute an embedded operating system, and include services provided by the operating system that can be used for file system manipulation, display & audio generation, basic networking, firewalling, data encryption and communications.

As used in this specification and the claims, the singular forms “a,” “an,” and “the” refer to plural referents unless the context clearly dictates otherwise. For example, reference to “the arm 50” in the singular includes a plurality of arms 50, and reference to “the electrode 104” in the singular includes one or more electrodes 104 and equivalents known to those skilled in the art. Thus, in various embodiments, any reference to the singular includes a plurality, and any reference to more than one component can include the singular.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein.

It is, therefore, to be understood that the foregoing embodiments are presented by way of example only and that, within the scope of the appended claims and equivalents thereto, inventive embodiments may be practiced otherwise than as specifically described and claimed. Illustrative embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure. Disclosed embodiments, or portions thereof, may be combined in ways not listed above and/or not explicitly claimed. Thus, one or more features from variously disclosed examples and embodiments may be combined in various ways. For example, it is to be understood that the present disclosure contemplates that, to the extent possible, one or more features of any embodiment can be combined with one or more features of any other embodiment.

Various inventive concepts may be embodied as one or more methods, of which examples have been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Although the above discussion discloses various exemplary embodiments of the invention, it should be apparent that those skilled in the art can make various modifications that will achieve some of the advantages of the invention without departing from the true scope of the invention.

The embodiments of the invention described above are intended to be merely exemplary; numerous variations and modifications will be apparent to those skilled in the art. Such variations and modifications are intended to be within the scope of the present invention as defined by any of the appended claims.

What is claimed is:

1. A method stimulating a nerve, the method comprising:
providing a neuromodulation device having a channel that leads to a chamber having an electrode therein;
positioning a nerve at least partially inside the chamber;
stimulating the nerve with the electrode using a first set of parameters configured to provide an afferent control signal; and
stimulating the nerve with the electrode using a second set of parameters configured to provide an efferent control signal.

2. The method as defined by claim 1, wherein stimulating the nerve using the afferent sensory control signal is configured to treat overactive bladder or fecal incontinence.

3. The method as defined by claim 1, wherein the afferent sensory control signal has a frequency of between about 2 Hz and about 20 Hz.

4. The method as defined by claim 1, wherein the afferent sensory control signal has an amplitude of between about 0.4 mA and about 1.0 mA.

5. The method as defined by claim 1, wherein the afferent sensory control signal has a pulse duration of between about 200 microseconds and about 300 microseconds.

6. The method as defined by claim 1, wherein a plurality of pulses defines a treatment session, the afferent sensory control signal having a treatment session time of between about 10 minutes and about 30 minutes, and a treatment session duty cycle of between about 10% and about 100%

7. The method as defined by claim 1, further comprising providing a stimulation treatment session 3 to 5 times per day.

8. The method as defined by claim 1, wherein the intensity of the efferent motor control signal is sub-threshold, and stimulating the nerve using the efferent motor control signal is configured to treat stress urinary incontinence or fecal incontinence.

9. The method as defined by claim 8, wherein the efferent motor control signal has a frequency of between about 40 Hz and about 80 Hz.

10. The method as defined by claim 1, wherein the efferent motor control signal has a sub-threshold amplitude of between about 0.5 mA and about 2 mA.

11. The method as defined by claim 1, wherein the efferent motor control signal has a pulse duration of between about 250 microseconds and about 400 microseconds.

12. The method as defined by claim 1, wherein a plurality of pulses defines a treatment session, the efferent sensory control signal having a treatment session time of between about 10 minutes and about 13 minutes, and a treatment session duty cycle of between about 10% and about 100%

13. The method as defined by claim 1, wherein the nerve is the perineal nerve

14. The method as defined by claim 1, wherein the nerve is the inferior rectal nerve.

15. A method of stimulating a nerve to treat stress urinary incontinence, the method comprising:
providing a neuromodulation device having a channel that leads to a chamber having an electrode therein;
stimulating a perineal nerve positioned at least partially inside the chamber with the electrode using an SUI control signal, the SUI control signal having a frequency of between about 50 Hz and about 80 Hz, a threshold stimulation pulse of between about 0.5 mA and 2.0 mA, a pulse duration of between about 250 microseconds and about 400 microseconds, a total SUI session time of between about 10 minutes and about 13 minutes, the total SUI session having a duty cycle of between about 5% and about 15%.

16. The method of claim 15, wherein stimulating the perineal nerve treats SUI.

17. The method of claim 15, further comprising stimulating the perineal nerve positioned at least partially inside the chamber with the electrode OAB control signal after stimulation the perineal nerve using the SUI control signal, the OAB control signal having a frequency of between about 2 Hz and about 20 Hz, a sub-threshold stimulation pulse of between about 0.4 mA and 1.0 mA, a pulse duration of between about 200 microseconds and about 300 microseconds, total OAB session time of between about 10 minutes and about 30 minutes, the total OAB session having a duty cycle of between about 100% and about 10%.

18. The method of any of claim 15, wherein the stimulation pulse is bi-phasic.

19. A method of stimulating a nerve to treat overactive bladder, the method comprising:
providing a neuromodulation device having a channel that leads to a chamber having an electrode therein;
stimulating a perineal nerve positioned at least partially inside the chamber with the electrode using an OAB control signal, the OAB control signal having a frequency of between about 2 Hz and about 20 Hz, a sub-threshold stimulation pulse of between about 0.4 mA and 1.0 mA, a pulse duration of between about 200 microseconds and about 300 microseconds.

20. A method of stimulating a nerve to treat fecal incontinence, the method comprising:
providing a neuromodulation device having a channel that leads to a chamber having an electrode therein;
stimulating the inferior rectal nerve positioned at least partially inside the chamber with the electrode using an efferent FI control signal, the efferent FI control signal having a frequency of about 50 Hz and about 80 Hz, a stimulation pulse of between about 0.5 mA and about 2.0 mA, a pulse duration of between about 200 microseconds and about 300 microseconds, a total efferent FI session time of between about 10 minutes and about 30 minutes, the total efferent FI session having a duty cycle of between about 100% and about 10%.

21. The method of claim 20, further comprising stimulating the inferior rectal nerve positioned at least partially inside the chamber with the electrode using an afferent FI control signal having a frequency of between about 2 Hz and about 20 Hz, a sub-threshold stimulation pulse of between about 0.4 mA and 1.0 mA, a pulse duration of between about 200 microseconds and about 300 microseconds.

22. The method of claim 21, wherein the total afferent FI session time of between about 10 minutes and about 30 minutes, the total afferent FI session having a duty cycle of between about 100% and about 10%.

23. The method of claim 21, further comprising:
stimulating the inferior rectal nerve positioned at least partially inside the chamber with the electrode using an OAB control signal, the OAB control signal having a having a frequency of between about 2 Hz and about 20 Hz, a sub-threshold stimulation pulse of between about 0.4 mA and 1.0 mA, a pulse duration of between about 200 microseconds and about 300 microseconds.
